SEQUENCE LISTING (1 sequence)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

(19) United States
(12) Patent Application Publication
(10) Pub. No.: US 2019/0008918 A1
(43) Pub. Date: Jan. 10, 2019

(54) IMMUNOMODULATION THERAPIES FOR CANCER

(71) Applicant: BIOXCEL CORPORATION, Branford, CT (US)

(72) Inventors: Sanatan UPMANYU, Gurgaon (IN); Snigdha GUPTA, Gurgaon (IN); Sameer SHARMA, Himachal Pradesh (IN); Luca RASTELLI, Norwell, MA (US); Krishnan NANDABALAN, Branford, CT (US)

(21) Appl. No.: 16/081,144

(22) PCT Filed: Mar. 8, 2017
(86) PCT No.: PCT/US17/21400

§ 371 Date: Aug. 30, 2018

Related U.S. Application Data
(60) Provisional application No. 62/305,052, filed on Mar. 8, 2016.

(51) Int. Cl. A61K 38/06; A61K 45/06; A61P 35/00

(52) U.S. Cl. CPC A61K 38/06; A61P 35/00; A61K 45/06

(57) ABSTRACT

The present disclosure provides a novel combination of a NOD2 agonist with an immunotherapeutic agent for the treatment of cancer. The disclosure also comprises methods of treatments wherein a pharmaceutical composition of NOD2 agonist is used in combination with an immunotherapeutic agent. A pharmaceutical composition comprises of NOD2 agonist and an immunotherapeutic agent comprising PD-1 axis antagonist or CTLA4 antagonist with a pharmaceutically acceptable diluent or carrier.

### FIELD OF THE INVENTION

The present disclosure is in the field of immuno-oncology. More specifically, the present disclosure relates to novel combinations of NOD2 agonists and immunotherapeutic agents. The disclosure also provides methods of treating cancer and kits containing pharmaceutical compositions.

### CROSS REFERENCE TO RELATED APPLICATION

This application claims priority to U.S. Provisional Application No. 62/305,052, filed on Mar. 8, 2016, the disclosure of which is herein incorporated by reference in its entirety for all purposes.

### BACKGROUND OF THE INVENTION

Cancer involves abnormal cell growth and it behaves differently depending on whether it is non-cancerous (benign) or cancerous (malignant). Chemotherapeutic agents are used for the treatment of cancer but an effective treatment option is not available. Among them, NOD2 agonist is one of the chemotherapeutic agents.

NOD2 is a member of the Nucleotide-binding oligomerization domain like receptors (NLR) family of cytoplasmic pathogen recognition receptors that detect bacterial motifs, in particular muramyl-di-peptide, a bacterial cell wall component. NOD2 agonists include Muramyl dipeptide, Muramyl tripeptide (Mifamurtide), Murabutide. Romurtide, or Muramyl tetrapeptide.

Mifamurtide is a NOD2 agonist and a fully synthetic derivative of muramyl dipeptide. It stimulates macrophages and monocytes to seek out and destroy cancer cells. It increases the levels of β2-microglobulin, TNF-α, IL-1β, IL-1 receptor, IL-6, neopterin, ceruloplasmin, and C-reactive protein.

Mifamurtide is disclosed in U.S. Pat. No. 4,406,890 i.e. assigned to Novartis Corporation. It is available as liposomal formulation (Mepact®) as disclosed in U.S. Pat. No. 4,971,802. The other patents on formulations of Mifamurtide are EP Patent No. 1,909,758; U.S. Pat. No. 7,641,911 and U.S. Pat. No. 5,334,583, wherein Mifamurtide was formulated as lyophilized powder, oil in water emulsion, and non-suppository topical composition, respectively.

Further, the combination of Mifamurtide with α-interferon polypeptide for the treatment of an infection caused by viruses is disclosed in U.S. Pat. No. 5,137,720. However, the present invention provides entirely different combination of NOD2 agonist and immunotherapeutic agent for the treatment of cancer whereas U.S. Pat. No. '720 for the treatment of viral infection.

U.S. Pat. No. 4,414,204 and EP Patent Application No. 0056560 disclose the combination of antibiotics with muramyl peptide derivatives such as Mifamurtide to enhance the activity of antibiotics. However, the present invention provides an anticancer combination of NOD2 agonist and immunotherapeutic agent whereas U.S. Pat. No. '204 and EP'560 patents does not disclose about the cancer and specific for increasing the activity of antibiotics.

FR Patent No. 2,703,251 discloses the combination of cytokines such as interferon-7, transforming growth factor-beta (TGF-beta) and interleukin-6 (IL-6) with Mifamurtide for the treatment of infections and other condition including cancers that have inflammation as one of the pathological features. However, the present invention provides combinations of NOD2 agonists and immunotherapeutic agents to treat cancer where the cancer has immune suppression and thus combination will bring about immune stimulation by acting against immune suppressive cytokines such as TGF-beta, as well as causes the upregulation of immune-stimulatory cytokines such as IL-6 or IFN-γ and does not need the addition of any other cytokines.

U.S. Pat. No. 5,877,147 discloses the use of Mifamurtide in skin cancer associated with other cancers such as cervical cancer, colorectal cancer, melanoma, and gastric cancer. However, the present invention provides improved methods with better clinical outcomes for the treatment of cancer by administration of a combination comprising NOD2 agonist and an immunotherapeutic agent whereas U.S. Pat. No. '147 just limited to the treatment of skin-specific precancerous lesions leads to skin cancer.

Hewitt R. E. et al., Clinical immunol. 2012, 143, 162-169 discloses that the peptidyl component of Mifamurtide (MDP) to stimulate monocytic cells in-vitro to enhance the expression of PD-L1. This prior art however, shows the effect in-vitro and in monocytes from healthy subjects at a fixed dose of 10 μg/ml of MDP. This information is only confined to healthy subjects and does not guarantee the observation in subjects with cancer experiencing immune suppression.

Immuno-oncology is an area of medicine that focuses on the development of therapies that improve the body's ability to generate immune responses against cancer. It is one of the most promising and fastest growing areas of cancer research. Immune-checkpoints refer as molecules that needs to be activated (stimulatory or costimulatory checkpoint molecules) or inactivated (inhibitory checkpoint molecules) to start immune responses via interaction between specific receptors and ligand pairs. Hence, immune checkpoint inhibitor(s) play a crucial role in the treatment of the cancer.

Currently, immune-checkpoints such as PD-1, PD-L, PD-L2, and CTLA4 are targeted for the treatment of cancer. There are many immune-checkpoint inhibitors in the clinical trials.

However, these immunotherapies are not effective in the few segment of cancers (such as non-responsive cancer), for example PD-1 or PD-L1 inhibitors immunotherapy as mentioned in Topalian S. L. et al., The New England Journal of Medicine, 2012; 366 (26), 2443-2454; and Brahmer J. R et al., The New England Journal of Medicine, 2012; 366, (26), 2455-2465; and Xia Bu et al. Trends in Molecular medicine, June 2016, 22 (6), 448-451. Xia Bu et al. discloses most of the patients fail to respond to PD-1 pathway blockade and the present combination targeting produces effective anti-tumor immunotherapy.

Accordingly, there is a need to develop combination therapies to initiate or enhance the efficacy of immunotherapeutic agents. Hence, the present disclosure provides novel approaches to overcome the problems in current immunotherapies as outlined above and to provide combination therapies of chemotherapeutics agents such as NOD2 agonists (e.g. Mifamurtide) and immunotherapeutic agents such as PD-1 axis antagonist or CTLA4 antagonist to convert the non-responsive cancer to a responsive cancer. The present disclosure thus relates to combination therapies to initiate, enhance, or improve anti-tumor immune responses that enable, enhance, or improve the subject's tumor responses to immunotherapeutic agents.

### SUMMARY OF THE INVENTION

The present disclosure provides that NOD2 agonists interact and thereby enhance the activity of certain immunotherapeutic agents that have different mechanisms of action, for the treatment of cancer. In some instances, the surprising enhancement can be associated with the combination of NOD2 agonists with immunotherapeutic agents that can act synergistically to treat cancer. It has been surprisingly found that NOD2 agonist such as Mifamurtide causes upregulation of PD-L1 and thereby enhances anti-cancer effect of PD-1 axis antagonist. Therefore, the present disclosure provides the compositions and methods of treating cancer in subjects that include administering to the subjects a combination therapy of a NOD2 agonist and an immunotherapeutic agent, wherein the immunotherapeutic agent comprises a PD-1 axis antagonist.

The anticancer effect of combining a NOD2 agonist such as Mifamurtide with an immunotherapeutic agent such as PD-1 antagonist was found to be greater than expected based on the response observed when the agents were used alone. Without being bound by theory, it is thought that Mifamurtide may act via a different mechanism in comparison to the PD-1 antagonist on the cancerous tissues. Moreover, in advanced stages of cancer where immunotherapy is refractive or non-responsive, combination therapies can be more effective on the cancer cells due to different mechanism of Mifamurtide and can also enhance the effects of the immunotherapy.

The present disclosure provides methods of treating subjects having cancer comprising administering to the subject in need thereof a therapeutically effective amount of NOD2 agonist and an immunotherapeutic agent. In particular, the subject has been determined to have cancer that is refractive or non-responsive.

In further aspect, the present disclosure provides combination therapies that can include NOD2 agonists such as Mifamurtide, and immunotherapeutic agents that can include PD-1 axis antagonists (e.g. a PD-1 antagonist, a PD-L1 antagonist, and a PD-L2 antagonist) or CTLA4 antagonists. In some aspects, the NOD2 antagonists can further include Murabutide, Muramyl tetrapeptide, Muramyl tripeptide, Muramyl dipeptide, Romurtide, M-TriDaP (N-acetyl-muramyl-L-Ala-γ-D-Glu-meso-diaminopimelic acid), N-Glycolyl Muramyldipeptide, M-Tri_{LYS} (MurNAc-Ala-D-isoGln-Lys), MDP(D-Glu²)-OCH₃, Glucosaminyl muramyldipeptide, and any combinations thereof. In some aspects, the PD-1 antagonists can include ANA011, AUNP-12, BGB-A317, KD033, Pembrolizumab, MCLA-134, mDX400, MEDI0680, muDX400, Nivolumab, PDR001, PF-06801591, REGN-2810, SHR-1210, STI-A1110, TSR-042, ANB011, and XCE853. In some aspects, the CTLA4 antagonists can include KAHR-102, ABR002, KN044, Tremelimumab, and Ipilimumab.

In another aspect, the present disclosure provides improved methods of treating subjects who are already on immunotherapeutic agents for the treatment of cancer. The improvement can include administering effective amounts of NOD2 agonists to the subjects in combination with the immunotherapeutic agents to enhance the anti-cancer effects of the immunotherapeutic agents.

In another aspect, the present disclosure provides methods of enhancing immune function in subjects having cancer. In some aspects, the methods can comprise administering therapeutically effective amounts of a combination of a NOD2 agonist and an immunotherapeutic agent. In some aspects, the NOD2 agonist can be Mifamurtide and the immunotherapeutic agent can be PD-1 axis antagonists or CTLA4 antagonists.

In another aspect, the present disclosure provides pharmaceutical compositions that comprise a NOD2 agonist, an immunotherapeutic agent, and one or more pharmaceutically acceptable carrier or adjuvant thereof. In some aspects, the NOD2 agonist is Mifamurtide.

In yet another aspect, the PD-1 antagonist can be administered at a dose of about 0.1 mg/kg to about 10 mg/kg of body weight. In one aspect, the PD-1 antagonist can be administered at a dose of about 2 mg/kg to about 5 mg/kg of body weight once every two weeks or once every three weeks. In another aspect, the PD-L1 antagonist can be administered at a dose of about 1 mg/kg to about 20 mg/kg of body weight once every three weeks. In certain aspects, PD-L2 antagonist can be administered at a dose of about 0.3 mg/kg to about 30 mg/kg of body weight once every two weeks. In certain other aspect, the NOD2 agonist can be administered at a dose of about 0.01 mg/kg to about 1.5 mg/kg of body weight. In one aspect, the NOD2 agonist can be administered at a dose of about 0.01 mg/kg to about 0.5 mg/kg of body weight. In another aspect, the NOD2 agonist can be administered at a dose of about 0.03 mg/kg to about 0.2 mg/kg of body weight twice weekly or once weekly.

In some aspects, the NOD2 agonists and the immunotherapeutic agents can be administered simultaneously to the subjects. In some aspects, the NOD2 agonists and the immunotherapeutic agents can be administered sequentially in either order. In some aspects, the combination of the NOD2 agonist and the immunotherapeutic agent can be administered for as long as clinical benefit is observed or until unmanageable toxicity or disease progression occurs. In some aspects, the NOD2 agonist and the immunotherapeutic agent can be administered concurrently in separate compositions. In some embodiments, the subject is human.

In some aspects, the PD-1 antagonists can be Pembrolizumab or Nivolumab. In some aspects, the PD-L1 antagonists can be Durvalumab, Atezolimumab. or Avelumab. In some aspects, the PD-L2 antagonists can be AMP-224 or rHIgM12B7.

In some aspects, the NOD2 agonists can be administered intravenously. In some aspects, the PD 1 axis antagonists or CTLA4 antagonists can be administered intravenously.

In some aspects, the cancer as described herein can be refractory cancers. In some aspects, the cancer can be colorectal cancer, melanoma, osteosarcoma, head and neck cancer, gastric cancer, breast cancer (triple negative breast cancer), acute lymphoblastic leukemia (ALL), non-small cell lung cancer (NSCLC), ovarian cancer, hepatocellular cancer, pancreatic cancer, renal cell cancer, and/or bladder cancer. In one aspect, the cancer is colorectal cancer.

In a further aspect, the present disclosure provides kits for treating subjects having cancer. In some aspects, the kits comprise (a) a dosage of a NOD2 agonist ranging from 0.01 mg/Kg to about 1.5 mg/Kg of body weight, (b) a dosage of a PD-1 axis antagonist ranging from 0.1 mg/Kg to about 30 mg/Kg of body weight, and (c) a package insert for either simultaneously or sequentially administering the NOD2 agonist and the PD-1 axis antagonist for treating the subjects.

The present disclosure also provides methods of enhancing proinflammatory cytokines production in a human having a tumor. In some aspects, the methods can include administering therapeutically effective amounts of (i) a Mifamurtide and (ii) an immunotherapeutic agent to the human. In some aspects, the combination of the Mifamurtide and the immunotherapeutic agent can provide a synergistic increase in proinflammatory cytokines production. In some aspects, the immunotherapeutic agent can be a PD-1 antagonist, a PD-L1 antagonist, a PD-L2 antagonist, or a CTLA4 antagonist.

The present disclosure also provides methods of inducing apoptosis in a human having a tumor. In some aspects, the methods can include administering therapeutically effective amounts of (i) a Mifamurtide and (ii) an immunotherapeutic agent to the human. In some aspects, the combination of the Mifamurtide and the immunotherapeutic agent provides a synergistic increase in apoptosis. In some aspects, the immunotherapeutic agent can be a PD-1 antagonist, a PD-L1 antagonist, a PD-L2 antagonist, or a CTLA4 antagonist.

This summary provides examples of the invention which are not intended to be limiting on the scope of the invention. The features of the invention described above and recited in the claims may be combined in any suitable manners. The combinations described herein and recited in the claims are not intended to limit the scope of the invention.

### BRIEF DESCRIPTION OF THE DRAWINGS

FIG. 1 shows the therapeutic efficacies of Mifamurtide (Source: Sigma, SML0195) alone and in combination with a PD-1 antagonist (Catalogue no. BE0146, BioXcell) in MC38 mouse model of colon adenocarcinoma. The combination therapies in both dosing regimens (Mifamurtide 30 μg biw+PD-1 antagonist 5 mg/Kg biw and Mifamurtide 10 μg biw+PD-1 antagonist 5 mg/Kg biw) showed synergistic effects on reducing the tumor volumes.

### DETAILED DESCRIPTION OF THE INVENTION

The present invention will become fully understood from the detailed description given below. However, the detailed description and the specific embodiments are illustrations of desired embodiments of the present invention, and are described only for an explanation. While the detailed description concludes with claims particularly pointing out and distinctly claiming that, which is regarded as the invention, it is anticipated that the invention can be more readily understood through reading the following detailed description of the invention and study of the included examples. Various possible changes and modifications will be apparent to those of ordinary skill in the art based on the detailed description.

### Abbreviations

A2AR (adenosine A_{2A} receptor)

### APC (Antigen Presenting Cells)

### B7H1 (B7 Homolog 1)

b.i.w. or biw (twice in a week or twice weekly)
CARD 15 (Caspase recruitment domain-containing protein)

### CCR4 (Chemokine (C-C Motif) Receptor 4)

CD (Cluster of differentiation)
CTLA4 (Cytotoxic T-lymphocyte-associated protein 4)
DMEM (Dulbccco's modified eagle's medium)
IBD1 (Inflammatory bowel disease protein 1)
IDO (Indoleamine-pyrrole 2, 3-dioxygenase)

### IFN-γ (Interferon-gamma)

### IL (Interleukin)

GM-CSF (Granulocyte-macrophage colony-stimulating factor)
LAG3 (Lymphocyte-activation gene 3)
MCP-1 (Monocyte chemotactic protein 1)
MIP-2 (Macrophage inflammatory protein-2)
MC38 (Murine colon adenocarcinoma cell line)
MDP (Muramyl dipeptide)
MTP (Muramyl tripeptide)
MDSCs (Myeloid-derived suppressor cells)
NOD2 (Nucleotide-binding oligomerization domain-containing protein 2)
NLR (Nucleotide-binding oligomerization domain like receptors)
PD-1 (Programmed death 1)
PD-L (Programmed cell death ligand)
PGD₂ (Prostaglandin D₂)
PGE₂α (Prostaglandin E₂alpha)
Q1W (Once a week)
Q2W (Once every two weeks)
Q3W (Once every three weeks)
Q4W (Once every four weeks)

### SEM (Standard Error of Mean)

TIM3 (T-cell immunoglobulin and mucin-domain containing-3)
TGF-beta (Transforming growth factor-beta)

### TGI Tumor Growth Inhibition

### TNF (Tissue Necrosis Factor)

The details of the present invention are as follows:

### I. Definitions

Various terms are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

As used herein, the term “subject” refers to an animal, preferably a mammal such as non-primate (e.g. cows, pigs, horses, cats, dogs, rats, etc.) and a primate (e.g., monkey and human), and most preferably a human.

As used herein, the term “cancer” can be used interchangeably with “tumor” or “carcinoma” or “neoplasm”. The term “cancer” refers to the physiological condition in subjects that is typically characterized by unregulated or hyperproliferative cell growth. It includes a wide variety of tumor types, including both solid tumors and non-solid tumors such as leukemia and lymphoma. Carcinomas, sarcomas, myelomas, lymphomas, leukemia, or cancers having mixed types can all be treated using the methods and compositions as described in the present disclosure. Compounds and methods of the present disclosure can inhibit and/or reverse undesired hyperproliferative cell growth involved in such conditions.

As used herein, the term “effective amount” or “therapeutically effective amount” of a compound refers to a nontoxic but a sufficient amount of the compound to provide the desired therapeutic or prophylactic effect to patients or individuals in need thereof. In the context of treatment of cancer, a nontoxic amount does not necessarily mean that an agent is not used, but rather means the administration of a tolerable and sufficient amount to provide the desired therapeutic or prophylactic effect to a patient or an individual. The effective amount of a pharmacologically active compound may vary depending on the route of administration, as well as the age, weight, and sex of the individual to which the drug or pharmacologically active agent is administered. Metabolism, bioavailability, and other factors that affect plasma levels of a compound following administration within the unit dose ranges disclosed further herein for different routes of administration can also be evaluated to determine appropriate and effective amounts of the compounds.

As used herein, the term “NOD2 agonist” refers to a substance binds to a Nucleotide-binding oligomerization domain-containing protein 2 (NOD2) receptor to activate NOD2. NOD2 agonists can include, but are not limited to Murabutide, Mifamurtide, Muramyl tetrapeptide, Muramyl tripeptide. Muramyl dipeptide. Romurtide, M-TriDaP (N-acetyl-muramyl-L-Ala-γ-D-Glu-meso-diaminopimelic acid), N-Glycolyl Muramyldipeptide, M-TriLYS (MurNAc-Ala-D-isoGln-Lys), MDP(D-Glu2)-OCH3. Glucosaminyl muramyldipeptide, or any derivatives thereof.

As used herein, the term “Mifamurtide” refers to Mifamurtide free base or its pharmaceutically acceptable salts (e.g. sodium salt of Mifamurtide) or any other salts or its pharmaceutically acceptable solvates or its pharmaceutically acceptable derivatives thereof. It can be used as agonist that activates NOD2 pathway. The alternative names of Mifamurtide include muramyl tripeptide phosphatidylethanolamine, MTP-PE, Juvonen. and CGP 19835A.

The term “treating” or “treatment of cancer” can be used interchangeably, within the context of the instant invention, means an alleviation of symptoms associated with a disorder or a disease, or halt of further progression or worsening of those symptoms, or prevention or prophylaxis of the disease or the disorder. For example, within the context of treating patients in relation to the NOD2 agonists and the immunotherapeutic agents, successful treatment may include a reduction in tumor adhesion and anchorage; an alleviation of symptoms related to a cancerous growth of tumor, or proliferation of diseased tissue; a halt in the progression of a disease such as cancer or in the growth of cancerous cells. Treatments may also include administering the pharmaceutical formulations of NOD2 agonists in combination with the immunotherapeutic agents. The pharmaceutical formulations may be administered before, during, or after surgical procedures and/or radiation therapy. In this invention, NOD2 agonists and the immunotherapeutic agents can be co-administered into the subject that leads to an improved prognosis.

As used herein the term, “administering” or “administration” refers to the physical introduction of a composition comprising a therapeutic agent to a subject, using any of the various methods and delivery systems known to those skilled in the art. Preferred routes of administration for the PD-1 antagonists, PD-L1 antagonists, PD-L2 antagonists, CTLA4 antagonists, and NOD2 agonists can include, but are not limited to intravenous, intramuscular, subcutaneous, intraperitoneal, spinal, or other parenteral routes of administration such as by injection or infusion.

The phrase “parenteral administration” as used herein means modes of administration other than enteral and topical administration, usually by injection, and can includes, without limitation, intravenous, intramuscular, intra-arterial, intrathecal, intralymphatic, intralesional, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion, as well as in vivo electroporation. As used herein, “intravenous administration” refers to delivery of a therapeutic directly into a vein.

The terms “co-administration” or “co-administering” as used herein, encompass administration to a subject a combination of a NOD2 agonist and an immunotherapeutic agent. Co-administration includes simultaneous administrations in separate compositions or administrations at different times in separate compositions. Simultaneous or sequential administrations in separate compositions are preferred.

The term “pharmaceutical composition” as used in accordance with the present disclosure relates to compositions that can be formulated in any conventional manners using one or more pharmaceutically acceptable carriers or excipients.

As used herein, “pharmaceutically acceptable carrier” refers to a carrier medium which does not interfere with the effectiveness of the biological activity of the therapeutic ingredients and is not toxic to the subject.

As used herein, the term “antagonist” refers to a molecule which blocks (reduces or prevents or decreases) a biological activity.

As used herein, the term “enhanced” refers to allowing a subject or tumor cell to improve its ability to respond to a treatment as disclosed herein.

As used herein and in its broadest sense, the terms “synergistically” or “synergistic effect” or “synergizes” refer to a phenomenon where treatment of patients with a combination of therapeutic agents (e.g. a NOD2 agonist in combination with a PD-1 antagonist) manifests a therapeutically superior outcome to the outcome achieved by each active ingredient of the combination used at its optimum dose.

The present invention describes the use of various therapeutic agents as described below:

### II. Therapeutic Agents

A. NOD2 Agonist:

The present invention discloses that an immune escape mechanism could be targeted in combination with various therapeutic agents or targets.

One of the targets is Nucleotide-binding oligomerization domain like receptors (NLR) family of cytoplasmic pathogen recognition receptors that detect bacterial motifs, particularly the bacterial cell wall component, muramyl dipeptide.

NLR family includes subfamilies such as Nucleotide-binding Oligomerization Domain protein 2 (NOD2). NOD2 is also known as caspase recruitment domain-containing protein 15 (CARD 15) or inflammatory bowel disease protein 1 (IBD1). Its target is expressed in peripheral blood leukocytes. It is mainly in the antigen presenting cells including the dendritic cells, monocytes, macrophages as well as in certain epithelial cell. NOD2 recognizes the muramyl dipeptide (MDP) component of invading microbes and induces the innate immune response by the NF-kappaB inflammatory pathway.

NOD2 agonists of the present disclosure can include, but are not limited to Mifamurtide, Murabutide. M-TriDaP (N-acetyl-muramyl-L-Ala-γ-D-Glu-meso-diaminopimelic acid), N-Glycolyl Muramyldipeptide, M-TriLYS (MurNAc-Ala-D-isoGln-Lys), MDP(D-Glu2)-OCH3, Glucosaminyl muramyl dipeptide, Muramyl dipeptide (MDP), Muramyl tripeptide, Muramyl tetrapeptide, Romurtide (L18MDP), or any derivatives thereof.

Mifamurtide is a synthetic molecule that can have the capability to stimulate the mammalian innate immune system. It is a synthetic analogue of peptidoglycan component of the gram positive and gram negative bacterial cell wall called muramyl dipeptide (MDP). Mifamurtide is a conjugate of muramyl tripeptide and the tripeptide moiety is linked to dipalmitoyl phosphatidyl ethanolamine via an alanine moiety. This phospholipid facilitates the incorporation of the peptides into the liposomes.

The present disclosure shows that when administered in a mouse model of colon adenocarcinoma. Mifamurtide can cause the upregulation of the proinflammatory cytokines while also causing the tumor regression. Due to the lipophilic property of the liposome and its peptidyl moiety, Mifamurtide can activate monocyte/macrophage through the NOD2-RIP2-NFkappaB pathway. This leads to the secretion of pro-inflammatory cytokines including TNF, interleukin (IL) such as IL-1, IL-6, IL-8, IL-12, IFN-γ, nitric oxide (NO), PGE_{2α}, and PGD₂, and the secretion of the chemokines as demonstrated in vivo that exhibits tumoricidal activity as observed in the present disclosure. The lipophilicity of Mifamurtide facilitates the passive transfer through the cytoplasmic membrane into cells that do not inheritably have the phagocytic property that macrophages or dendritic cells possess.

Without wishing to be bound by any specific theory or mechanism, it has been surprisingly found that Mifamurtide enhances the expression of PD-L1 as observed in this disclosure via secretion of IFN-γ, which increases the efficacy of the PD-1 antagonist. Mifamurtide in combination with the PD-1 axis antagonist increases the responsiveness of the existing PD-1 axis antagonist therapy in advanced refractory cancer, particularly when the tumors are not immune responsive. Therefore, the combination of Mifamurtide with PD-1 axis antagonist therapy can potentiate the efficacy and the outcome of the therapy by decreasing the tumor size, enhancing the tumor infiltration of tumoricidal effector cells via increasing the immune-stimulatory milieu and modulating the PD-L1 or PD-1 expression, and thereby reducing the refractiveness to the therapy.

Moreover, in these advanced stages of cancer where therapy gets refractive or non-responsive, the specific or selective expression of immune checkpoints by cancer cells creates an opportunity for endogenous cell-mediated and serologic immune attacks and for immunotherapeutic interventions.

B. Immuno-Therapeutic Agents:

As used herein, the term “immunotherapeutic agent” refers to any therapeutic approaches aimed at mobilizing or manipulating a subject immune system to treat or cure a disease, particularly cancer. It includes targeting tumour cells by recognizing the immunogenic proteins or antigens expressed by said tumour cells, which can be accomplished by utilizing either passively transferred immune molecules such as antibodies preparations designed to induce antibodies or T lymphocytes (T cells) recognizing a localized region of an antigen or an epitope specific to the tumor cell.

The immunotherapeutic agents can include, but are not limited to PD-1 axis antagonists (e.g. PD-1 antagonists, PD-L1 antagonists, and PD-L2 antagonists), CTLA4 antagonists, or any combinations thereof.

### PD-1 Axis Antagonist:

As used herein, the term “PD-1 axis antagonist” refers to a molecule that inhibits the interaction of a PD-1 axis binding partner with either one or more of its binding partner, so as to remove T-cell dysfunction resulting from signaling on the PD-1 signaling axis with a result being to restore or enhance T-cell function (e.g., proliferation, cytokine production, or target cell killing).

PD-1 axis antagonists can include, but are not limited to PD-1 antagonists (for example PD-1 antibody), PD-L1 antagonists (for example PD-L antibody), and PD-L2 antagonists (for example PD-L2 antibody).

As used herein, the terms “Programmed Death 1,” “Programmed Cell Death Protein 1,” “CD279,” “Cluster of Differentiation 279,” “Protein PD-1,” “PD-1,” “PDCD1,” “hPD-1,” and “hPD-1” are used interchangeably, and include variants, isoforms, species homologs of human PD-1, and analogs having at least one common epitope with human PD-1.

“PD-1 antagonists” means any chemical compounds or biological molecules that block the binding of PD-L1 expressed on a cancer cell to PD-1 expressed on an immune cell (T cell, B cell, or Natural Killer T cell) as well as block the binding of PD-L2 expressed on a cancer cell to the immune-cell expressed PD-1.

As used herein, the terms “Programmed Cell Death 1 Ligand 1,” “PD-L,” “PDCD1L1,” “PDCD1LG1, “CD274,” “Cluster of differentiation 274,” “B7 homolog 1,” “B7-H1,” and “B7H1” are used interchangeably, and include variants, isoforms, species homologs of human PD-L1, and analogues having at least one common epitope with human PD-L1.

As used herein, the terms “PD-L2” “Programmed death ligand 2,” “PD-1 ligand 2,” “B7 dendritic cell molecule,” “B7-DC.” “CD273,” “Cluster of differentiation 273,” “Programmed cell death 1 ligand 2,” “PDCD1 ligand 2,” and “PDCD1L2” are used interchangeably, and include variants, isoforms, species homologs of human PD-L2 and analogues having at least one common epitope with human PD-L2.

Programmed cell death protein 1 (PD-1) is a member of the CD28 family of receptors that also includes CTLA4. PD-1 is an immunoinhibitory receptor expressed on T-cells, B-cells, or myeloid cells but can be predominantly expressed on T-cells. It binds to two ligands, PD-L1 and PD-L2. PD-1 plays a critical role in immune response. Its engagement with PD-L1 and PD-L2 inhibits T cell proliferation and cytokine production such as IFN-γ, and enhances the development of cancer.

The amino acid sequence of human PD-1 can be found under GenBank Accession No. U64863. In some embodiments, the PD-1 antagonist binds to PD-1 protein of SEQ ID NO: 1 (Uniprot Accession Number Q15116).

PD-1 antagonists include, but are not limited to anti-PD-1 antibodies, antigen binding fragments thereof, immune adhesions, fusion proteins, oligopeptides, and other antagonists that also decrease, block, inhibit, abrogate or interfere with signal transduction resulting from the interaction of PD-1 with either PD-L1 or PD-L2. In one embodiment, PD-1 antagonist is a PD-1 antibody.

Suitable PD-1 antagonists include ANA011, AUNP-12, BGB-A317, KD033, Pembrolizumab. MCLA-134, mDX400, MEDI0680, muDX400, Nivolumab, PDR001, PF-06801591, REGN-2810, SHR-1210, STI-A110, TSR-042, ENUM 244C8, and XCE853. In a preferred embodiment, the PD-1 antagonist is Pembrolizumab (MK-3475, Lambrolizumab, Keytruda®, and SCH-900475) or Nivolumab (MDX-1106, MDX-1106-04. ONO-4538, BMS-936558, or Opdivo®). Nivolumaband Pembrolizumab are described in U.S. Pat. No. 8,008,449 and U.S. Pat. No. 8,345,509, respectively.

Other PD-1 antagonists are disclosed in U.S. Pat. No. 8,609,089; U.S. Pat. No. 7,488,802; U.S. Pat. No. 7,858,746; U.S. Pat. No. 8,618,757; U.S. Pat. No. 8,907,053; U.S. Pat. No. 8,993,731; U.S. Pat. No. 8,927,697; U.S. Pat. No. 8,735,553; U.S. Pat. No. 9,102,728; U.S. Pat. No. 9,181,342; U.S. Pat. No. 9,102,727; U.S. Pat. No. 9,029,315; U.S. Pat. No. 9,067,998; US 20100028330; US 20120114649; US 20130022629; US 20130017199; US 20150232555; US 20150203579; US 20150210769; US 20160319018; US 20160159905; US 20160272708; US 20160251436; US 20160075783; US 20160052990; CN 105175544, CN 104479020; CN 104761633; WO 2015058573; WO 2016077397; WO 2016015685; WO 2016197497 and WO 2016019270. In a specific embodiment, the PD-1 antagonist is Nivolumab (CAS Registry Number: 946414-94-4).

The present disclosure involves the use of a PD-1 antagonist (e.g., a PD-1 antibody) in combination with a selective NOD2 agonist for treating tumor or cancer. Accordingly, the PD-1 antagonist as disclosed herein binds to ligands of PD-1 and can interfere with, reduce, or inhibit the binding of one or more ligands to the PD-1 receptor, or can bind directly to the PD-1 receptor, without engaging in signal transduction through the PD-1 receptor.

PD-L1 antagonists can include, but are not limited to PD-L1 antibodies, antigen binding fragments thereof, immuno-adhesions, fusion proteins, and oligopeptides. Suitable PD-L1 antagonists include MPDL3280A (Atezolimumab or YW243.55.570 or RG&446), MDX-1105, MED14736 (Durvalumab), Avelumab (MSB0010718C), CA-170, CA-327, MCLA-145, SP142, STI-A1011, STIA1012, STI-A1010, STI-A1014, and KY1003. In a preferred embodiment, the PD-L antagonist is Avelumab, Durvalumab, or Atezolimumab. MDX-1105, Durvalumab, Atezolimumab, Avelumab and CA-170 are described in U.S. Pat. No. 7,943,743: U.S. Pat. No. 8,779,108; U.S. Pat. No. 8,217,149: US 20140341917: WO2015033301; and WO2015033299 respectively. Other PD-L antagonists are disclosed in U.S. Pat. No. 8,741,295; U.S. Pat. No. 8,552,154; US 20150355184 and US 20160108123.

PD-L2 antagonists can include, but are not limited to PD-L2 antibodies, antigen binding fragments thereof, immune adhesions, fusion proteins, and oligopeptides. In one embodiment, PD-L2 antagonist can be selected from the group comprising AMP-224, or rHIgM12B7. AMP-224, also known as B7-DCIg, is a PD-L2 Fc fusion soluble receptor described in WO2010027827 and WO2011066342.

The antibody or antigen binding fragment thereof, may be made using methods known in the art, for example, by a process comprising culturing a host cell containing nucleic acid encoding any of the previously described PD-1, PD-L1, or PD-L2 antibodies or antigen-binding fragment in a form suitable for expression, under conditions suitable to produce such antibody or fragment, and recovering the antibody or fragment.

In some embodiments, PD-1 axis antagonists can include, but are not limited to Nivolumab, Pembrolizumab, AMP-224, Atezolimumab, Durvalumab, and Avelumab. PD-1 axis antagonists (for example, PD-1 antibody) may be procured from BPS Biosciences and BioXcell.

### CTLA4 Antagonist:

Cytotoxic T-lymphocyte-associated antigen 4 (CTLA4), which is also known as CD152, is a protein involved in the regulation of the immune system. Suitable CTLA4 antagonists for use in the methods in the present disclosure may include, but are not limited to antibodies or fusion proteins or any fragments that target CTLA4 and antagonize the pathway of CTLA4. The suitable antibodies include, but are not limited to Ipilimumab, Tremelimumab, KAHR-102, AGEN 1884, ABR002, or KN044.

CTLA4 antibodies are described in U.S. Pat. No. 5,811,097; U.S. Pat. No. 5,855,887; U.S. Pat. No. 5,977,318; U.S. Pat. No. 6,051,227; U.S. Pat. No. 6,207,156; U.S. Pat. No. 6,682,736; U.S. Pat. No. 6,984,720; U.S. Pat. No. 7,034,121; U.S. Pat. No. 7,109,003: U.S. Pat. No. 7,132,281; U.S. Pat. No. 7,605,238; U.S. Pat. No. 8,697,845; U.S. Pat. No. 8,642,557: US 20090252741; US 20140105914; US 20160237154 and WO 2000037504. Other CTLA4 antibodies that can be used in the present methods can include, for example, those disclosed in Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998).

In a preferred embodiment, the CTLA4 antibody is Ipilimumab (also referred to as MDX010 or MDX101, CAS No. 477202-00-9, Yervoy® available from Medarex, Inc., Bloomsbury, N.J.) disclosed in U.S. Pat. No. 6,984,720; and Tremelimumab (also known as ticilimumab or CP-675,206) described in U.S. Pat. No. 6,682,736.

In some embodiments, the CTLA4 antagonist (e.g., CTLA4 antibody) can be used in combination of a selective NOD2 agonist for treating tumor or cancer.

### III. Method of Uses

The present disclosure provides methods for treating the conditions where enhanced immunogenicity is desired. A variety of tumors may be treated, or their progression may be delayed.

The present disclosure relates to the use of immunomodulating approaches in subjects by administering a combination therapy containing a NOD2 agonist and an immunotherapeutic agent such as PD-1 axis antagonist or CTLA4 antagonist for treating, ameliorating, or delaying the progression of tumor. It can be used in combination with other treatments, which include, but are not limited to surgery, radiation therapy, standard cancer chemotherapies, gene therapy, or vaccines. The combination therapies can improve T-cell priming, increase T cell stimulation, increase infiltrations of neutrophil and macrophages across tumor microenvironment, increase the activation of natural killer cells, enhance activation of dendritic cells, synergistically increase pro-inflammatory cytokines (e.g., IFN-γ. IL-6, IL-12p40, TNF-α) or chemokines (MCP-1 and MIP-2), decrease immune suppressive cells such as T regulatory cells and MDSCs, decrease tumor volumes, and reduce the toxicity.

The present disclosure also provides combination therapies for treating cancer. In some embodiments, a NOD2 agonist can be combined with an immunotherapeutic agent. The combination of Mifamurtide and PD-1 antagonist has been demonstrated herein (see Example 1) to produce early, durable antitumor activity in colorectal tumor bearing mice. Administering the combination therapies shows synergistic effects as compared to administering the PD-1 antagonist or Mifamurtide alone.

In one embodiment, the method is directed to administering pharmaceutical formulations that comprise effective amounts of a selective NOD2 agonist and an immunotherapeutic agent.

The present invention provides a novel combination approach comprising:
(i) an effective amount of a selective NOD2 agonist (for example Mifamurtide); and
(ii) an effective amount of an immunotherapeutic agent (for example PD-1 antagonist).

In certain embodiments, the present disclosure provides the combination therapy of a NOD2 agonist and an immunotherapeutic agent in a subject suffering from cancer and converts the ‘cold’ tumor type to the ‘hot’ tumor type that responds to the immunotherapeutic agent.

In some embodiments, the present disclosure provides methods of treating, preventing, or delaying cancer growth in subjects by administering to the subjects a combination of a NOD2 agonist and an immunotherapeutic agent. In certain embodiments, the NOD2 agonist is Mifamurtide and the immunotherapeutic agent is a PD-1 axis antagonist. In further embodiments, the PD-1 axis antagonist (PD-1 antagonist) is Opdivo® or Keytruda®. In further embodiments, the combination optionally comprises one or more other chemotherapeutic agent(s).

In some embodiments, the present disclosure provides methods of treating, preventing, or delaying growth of tumor in subjects by administering to the subjects a combination of Mifamurtide and an immunotherapeutic agent. In some embodiments, the immunotherapeutic agent can be selected from the group comprising of PD-1 antagonists, PD-L1 antagonists, PD-L2 antagonists, or CTLA4 antagonists. In some embodiments, the preferred PD-1 antagonist is Opdivo® or Keytruda®. In some embodiments, the preferred PD-L1 antagonist is Atezolimumab, Durvalumab, or Avelumab. In some embodiments, the preferred PD-L2 antagonist is AMP-224 or rHIgM12B7. In some embodiments, the preferred CTLA4 antagonist is Ipilimumab.

In some embodiments, the combination therapy increases the serum level of cytokine in the subject relative to the prior administration of the PD-1 axis antagonist.

In some embodiments, the present disclosure provides methods of enhancing or promoting immune response in subjects by administering to the subject a combination of a NOD2 agonist and an immunotherapeutic agent. In certain embodiments, the NOD2 agonist is Mifamurtide and the immunotherapeutic agent is PD-1 axis antagonist. In certain embodiments, the NOD2 agonist is Mifamurtide and the immunotherapeutic agent is CTLA4 antagonist. In further embodiments, the PD-1 axis antagonist is Opdivo® or Keytruda®. In some embodiments, the PD-1 axis antagonist (PD-L antagonist) is Atezolimumab, Durvalumab, or Avelumab. In some embodiments, the PD-1 axis antagonist (PD-L2 antagonist) is AMP-224 or rHIgM12B7.

In further embodiments, the combination therapy may also comprise at least one more additional therapeutic agent. The additional therapeutic agents may include, but are not limited to a chemotherapeutic agent other than NOD2 agonist, a biotherapeutic agent, an immunogenic agent (for example, attenuated cancerous cells, tumor antigens, antigen presenting cells such as dendritic cells pulsed with tumor derived antigen or nucleic acids), immune stimulating cytokines (for example, IL-2, IFNa2, GM-CSF), and cells transfected with genes encoding immune stimulating cytokines, which include, but are not limited to GM-CSF. The specific dosage and dosage schedule of the additional therapeutic agent can further vary, and the optimal dose, dosing schedule and route of administration can be determined based upon the specific therapeutic agent that is being used.

As described herein, the “chemotherapeutic agent” is a compound used for the treatment of cancer. The chemotherapeutic agents can include, but are not limited to alkylating agents such as dacarbazine, temozolamide (oral dacarbazine), procarbazine, thiotepa; nitrogen mustards such as cyclophosphamide, aldophosphamide, mechlorethamine, mechlorethamine oxide hydrochloride, chlorambucil, melphalan, chlomaphazine, estramustine, bendamustine, ifosfamide; novembichin, phenesterine, prednimustine; uramustine; ethylenimines and methylamelamines including altretamine, triethylenemelamine, triethylenephosphoramide, triethylenethiophosphoramide and trimethylolmelamine: alkyl sulfonates such as busulfan, improsulfan, and piposulfan; nitrosureas such as cannustine, semustine, streptozotocin, olaparib, chlorozotocin, fotemustine, lomustine, nimustine, ranimnustine, abiraterone: aziridines such as benzodopa, carboquone, meturedopa, and uredopa; anthracyclines derivatives such as daunorubicin, doxorubicin, esorubicin, rodorubicin, epirubicin, idarubicin, mitoxantrone, valrubicin, aclarubicin, detorubicin, marcellomycin, mitomycin C, azauridine, carmofur; taxane derivatives such as paclitaxel, albumin-engineered nanoparticle formulation of paclitaxel, docetaxel; epothilone derivatives such as epothilone; histone deacetylase inhibitors such as vorinostat, romidepsin; topoisomerase I inhibitors such as irinotecan, topotecan, silatecan, cositecan, exatecan, lurtotecan, gimatecan, belotecan, rubitecan; topoisomerase II inhibitors such as podophyllotoxin, etoposide, teniposide, tafluposide; kinase inhibitors such as bortezomib, erlotinib, gefitinib, imatinib, ceritinib, crizotinib, dabrafenib, vemurafenib, vismodegib, trametinib, sorafenib, sunitinib, lapatinib, axitinib, vandetinib, cabozatinib, lenvatinib, pazopanib; nucleotide analogs and precursor analogs such as azacytidine, azathioprine, capecitabine, arabinoside, cytarabine, doxifluridine, 5-fluorouracil (5-FU), gemcitabine, hydroxyurea, 6-mercaptopurine, methotrexate, tegafur, denopterin, pteropterin, trimetrexate, pemetrexed, pralatrexate, tioguanine (formerly thioguanine), fludarabine, floxuridine; ancitabine, dideoxyuridine, enocitabine; peptide antibiotics such as actinomycin, authramycin, bleomycin, peplomycin, cactinomycin, carminomycin, dactinomycin, puromycin, bestatin, zinostatin, zorubicin, pentostatin; platinum based antibiotics such as cisplatin and carboplatin; oxaliplatin; vinca alkaloids derivative such as vinblastine, vincristine, vindesine, vinorelbine; cannabinoid receptor antagonist such as delta-9-tetrahydrocannabinol; duocarmycin analogue such as adozelesin, bizelesin, carzelesin; mTOR inhibitors such as everolimus, temsirolimus; and others derivative including but not limited to mitotane, trilostane; pharmaceutically acceptable salts, acids or derivatives of any of the above; as well as combinations of two or more of the above.

### IV. Method of Administration

In some embodiments, suitable treatments can include administering to the subjects a therapeutic effective amount of a NOD2 agonist and an immunotherapeutic agent for the treatment of cancer or tumor.

In some embodiments, the present disclosure provides methods of treating cancer in subjects by adjunctive or combined administration (co-administration) includes simultaneous administration (i.e., in the same composition), concurrent administration (i.e., in a separate composition administered one right after the other in any order), or in sequentially in any order. Sequential administration is beneficial when therapeutic agents are in different dosage forms (one agent is tablet or capsule and another agent is sterile liquid) and/or in different dosing schedules. For instance, a NOD2 agonist can be administered twice weekly and an immunotherapeutic agent can be administered less frequently such as once every two weeks (Q2W), once every three weeks (Q3W), or once every four weeks (Q4W).

In some embodiments, the NOD2 agonist can be administered before the administration of an immunotherapeutic agent. In some embodiments, NOD2 agonist can be administered after the administration of an immunotherapeutic agent. In some embodiments, the NOD2 agonist can be administered concurrently with an immunotherapeutic agent.

NOD2 agonist in a combination therapy as described in the present disclosure may be administered by continuous infusion, or by doses at intervals of, e.g., daily (including twice or thrice a day dosing), every other day, three times per week, two time per week (b.i.w.), once a week (Q1W), once every two weeks (Q2W), once every three weeks (Q3W) or once every four weeks (Q4W), once a month, once every 3-6 months or longer preferably two time per week (b.i.w) and once a week. The NOD2 agonist can be administered at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, or about 1.5 mg/kg of body weight, inclusive of all ranges and subranges therebetween. In some embodiment, the dose of NOD2 agonist administered to prevent or treat cancer in a subject is administered in a dose ranging from about 0.01 mg/kg to about 1.5 mg/kg of body weight, from about 0.03 mg/kg to about 0.7 mg/kg of body weight, from about 0.1 mg/kg to about 0.9 mg/kg of body weight, from about 0.5 mg/kg to about 1.3 mg/kg of body weight, inclusive of all ranges and subranges therebetween. In a preferred embodiment, the dose of NOD2 agonist is from about 0.01 mg/kg to 0.5 mg/kg of body weight and more preferably at a dose range of about 0.03 mg/kg to 0.2 mg/kg of body weight. In some embodiments, the subject is a mammal. In one embodiment, the subject is a human.

In some embodiments, the dosing regimen of Mifamurtide can comprise administering the Mifamurtide to a subject at a dose range of about 0.03 mg/kg to about 0.2 mg/kg of body weight, about 0.05 mg/kg to about 0.1 mg/kg of body weight, about 0.04 mg/kg to about 0.07 mg/kg of body weight, about 0.06 mg/kg to about 0.09 mg/kg of body weight, about 0.08 mg/kg to about 0.2 mg/kg of body weight, inclusive of all ranges and subranges therebetween, when Mifamurtide is the NOD2 agonist in the combination therapy. In some embodiments, the intervals of the administration of Mifamurtide can be about two time per week (b.i.w.) for 12 weeks followed by once a week (Q1W) administration for 24 weeks and progress the similar cycle till tumor regression. In some embodiments, the subject is a human. In some embodiments, the NOD2 agonist can be administered for a total of 48 or 72 doses. In some embodiments, the NOD2 agonist can be administered with any doses on an as needed basis.

In some embodiments, the NOD2 agonist can be administered intratumorally at a dose of from about 0.6 to about 30 mg, from about 1 to about 25 mg, from about 5 to about 20 mg, from about 10 to about 15 mg, from about 0.8 to about 7 mg, from about 6 to about 17 mg, from about 11 to about 28 mg, inclusive of all ranges and subranges therebetween. In some embodiments, the NOD2 agonist is administered intratumorally preferably at a dose of from about 1.8 mg to about 12 mg (for 60 kg weight of the subject) twice a week for 12 weeks followed by once a week (Q1W) administration for 24 weeks and progress the similar cycle till tumor regression.

The dose frequency for administration of NOD2 agonist can be at an interval of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28 days, or once monthly for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, or about 36 weeks.

In some embodiments, the NOD2 agonist and an immunotherapeutic agent can be administered as separate formulations that have different dosing schedules. The separate formulations can be administered at a specific time interval, which can vary from 1 hour to 30 days. For example, one of therapeutic agents among them can be administered at an interval of about 30 days, about 29 days, about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, and/or about 1 day. In other examples, one of therapeutic agents among them can be administered at an interval of about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour from the administration of the other therapeutic agent.

In some embodiments, the immunotherapeutic agent can be administered one dose per day, one dose every 2 days, one dose every 3 days, one dose every 4 days, one dose every 5 days, once a week, once every two weeks (Q2W), once every three weeks (Q3W), once every four weeks (Q4W), once a month, once every 3 to 6 months or longer. In some embodiments, the immunotherapeutic agent is preferably administered once every two, three, or four weeks. In some embodiments, the immunotherapeutic agent is more preferably administered once every two or three weeks. In certain embodiments, the immunotherapeutic agent is administered as a single dose, in two doses, in three doses, in four doses, in five doses, or in six or more doses.

In other embodiments, the dosing regimen of a PD-1 antagonist as the immunotherapeutic agent in the combination therapy can be from about 0.1 mg/kg to about 10 mg/kg of body weight, about 0.5 mg/kg to about 9 mg/kg of body weight, about 1 mg/kg to about 8 mg/kg of body weight, about 2 mg/kg to about 7 mg/kg of body weight, about 3 mg/kg to about 6 mg/kg of body weight, about 4 mg/kg to about 5 mg/kg of body weight, about 0.3 mg/kg to about 4 mg/kg of body weight, about 0.6 mg/kg to about 6 mg/kg of body weight, about 1.5 mg/kg to about 7 mg/kg of body weight, about 3.5 mg/kg to about 10 mg/kg of body weight, inclusive of all ranges and subranges therebetween, with intra-patient dose escalation.

In some embodiments, the dosing regimen of a PD-1 axis antagonist as the immunotherapeutic agent in the combination therapy can be from about 1 mg/kg of body weight, about 2 mg/kg of body weight, about 3 mg/kg of body weight, about 5 mg/kg of body weight, about 10 mg/kg of body weight, or about 20 mg/kg of body weight. In some embodiments, the intervals of the administration can be about 14 days (±2 days) or about 21 days (±2 days) or about 30 days (±2 days) throughout the course of treatment.

In one preferred embodiment of the invention, the immunotherapeutic agent (for example PD-1 antagonist) in the combination therapy is Nivolumab, which is administered intravenously at a dose selected from the group consisting of: 1 mg/kg Q2W, 2 mg/kg Q2W, 3 mg/kg Q2W, 5 mg/kg Q2W, and 10 mg/kg Q2W.

In another preferred embodiment of the invention, the PD-1 antagonist in the combination therapy is Pembrolizumab or a Pembrolizumab variant, which is administered in a liquid medicament at a dose selected from the group consisting of 1 mg/kg Q3W, 2 mg/kg Q3W, 3 mg/kg Q3W, 5 mg/kg Q3W and 10 mg/kg Q3W, and flat-dose equivalents of any of these doses, i.e., such as 200 mg Q3W. In some embodiments, Pembrolizumab is provided as a liquid medicament which comprises 25 mg/ml Pembrolizumab, 7% (w/v) sucrose, 0.02% (w/v) polysorbate 80 in 10 mM histidine buffer (pH 5.5).

In some embodiments, the PD-1 antagonist is administered at a dose of about 2 mg/kg body weight at an interval of Q2W or Q3W. In some embodiments, the PD-L antagonist is administered at a dose of about 10 mg/kg, 15 mg/kg, or about 20 mg/kg of body weight at an interval of Q3W. In some embodiments, the PD-L2 antagonist is administered at a dose of about 0.3 mg/kg, about 10 mg/kg, or about 30 mg/kg of body weight at an interval of Q2W. In some embodiments, the CTLA4 antagonist is administered at a dose of about 1 to about 3 mg/kg of body weight at an interval of Q3W. In other embodiments, the CTLA4 antagonist can be administered at a dose of about 0.5 to about 4 mg/kg of body weight, about 1.5 to about 3.5 mg/kg of body weight, about 2 to about 3 mg/kg of body weight, about 1 to about 3.5 mg/kg of body weight, inclusive of all ranges and subranges therebetween.

The optimal dose for Pembrolizumab in combination with NOD2 agonist may be identified by dose escalation or dose de-escalation of one or both agents. In one embodiment, Pembrolizumab is administered at 200 mg or 2 mg/kg Q3W and the Mifamurtide is intravenously administered at a dose of from 1.8 to 12 mg (for 60 kg weight of the subject) twice a week (b.i.w.) for 12 weeks, followed by 1.8 to 12 mg (for 60 kg weight of the subject) once a week (Q1W) for 24 weeks. In one embodiment, a subject is treated with 200 mg of Pembrolizumab Q3W on Day 7 and treated with the Mifamurtide administered intravenously at a dose from 1.8 to 6 mg (for 60 kg weight of the subject) on Day 1, twice a week for 12 weeks, followed by a dose of from 1.8 to 12 mg (for 60 kg weight of the subject), once a week for 24 weeks. In another embodiment, Pembrolizumab is administered intravenously and administered until tumor regression or up to 36 weeks. In one embodiment, the subject is confirmed to have progressive disease while receiving prior PD-1 antagonist therapy.

The optimal dose for Nivolumab in combination with NOD2 agonist may be identified by dose escalation or dose de-escalation of one or both agents. In one embodiment, Nivolumab is administered at 240 mg or 3 mg/kg Q2W and the Mifamurtide is intravenously administered at a dose of from 1.8 to 12 mg (for 60 kg weight of the subject) twice a week (biw) for 12 weeks, followed by 1.8 to 12 mg (for 60 kg weight of the subject) once a week (Q1W) for 24 weeks. In another embodiment, Nivolumab is administered intravenously and administered until tumor regression or up to 36 weeks.

The dose frequency for administration of immunotherapeutic agent is usually at an interval of a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks. In some embodiments, the immunotherapeutic agent is administered at an interval of a period of about 2, 6, 7, 8, 9, 10, 11, 12, 13, 14, 15, 16, 21, 24, or 28 days, for example, 1 or 14 or 21 or 28 days.

In certain embodiments, the method of treatment of cancer comprises the co-administration of NOD2 agonist (for example Mifamurtide) and PD-1 axis antagonist with an administration cycle of 36 weeks till the tumor regresses. In some embodiments, the administration cycle comprises of the administration of NOD2 agonist to a subject in a dose range of 0.03 mg/kg to 0.2 mg/kg of body weight twice a week (b.i.w.) administration for 12 weeks followed by once a week (Q W) administration for 24 weeks and the administration of PD-1 axis antagonist of 0.1 mg/kg to 30 mg/kg on 7 day, once in three weeks (Q3W) and repeat the cycle till the tumor regresses, wherein the subject is a human.

In certain embodiments, the method of treatment of cancer comprises the co-administration of NOD2 agonist (for example Mifamurtide) and PD-1 axis antagonist with an administration cycle of 36 weeks till the tumor regresses, wherein the administration cycle comprises of the administration of NOD2 agonist in a dose range of 0.03 mg/kg to 0.2 mg/kg of body weight twice a week for 12 weeks followed by once a week administration for 24 weeks and the administration of PD-1 antagonist of about 2 mg/kg to about 5 mg/kg on day 7, once in two or three weeks and repeat the cycle till the tumor regresses.

In certain embodiments, the method of treatment of cancer comprises the co-administration of NOD2 agonist (for example Mifamurtide) and PD-1 axis antagonist with an administration cycle of 36 weeks till the tumor regresses, wherein the administration cycle comprises of the administration of NOD2 agonist to a subject in a dose range of 0.03 mg/kg to 0.2 mg/kg of body weight twice a week (b.i.w.) for 12 weeks followed by once a week (Q1W) or 24 weeks and the administration of PD-L1 antagonist of about 1 mg/kg to about 20 mg/kg of body weight on 7 day, once every three weeks (Q3W) and repeat the cycle till the tumor regresses, wherein the subject is human.

In certain embodiments, the method of treatment of cancer comprises the co-administration of a NOD2 agonist (for example Mifamurtide) and a PD-1 axis antagonist with an administration cycle of 36 weeks till the tumor regresses, wherein the administration cycle comprises of the administration of NOD2 agonist to a subject in a dose range of 0.03 mg/kg to 0.2 mg/kg of body weight twice a week for 12 weeks followed by once a week administration for 24 weeks and the administration of PD-L2 antagonist of about 0.3 mg/kg to about 30 mg/kg of body weight on 7 day, once every two weeks (Q2W) and repeat the cycle till the tumor regresses wherein the subject is human.

In certain embodiments, the method of treatment of cancer comprises the co-administration of NOD2 agonist (for example Mifamurtide) and CTLA4 antagonist with an administration cycle of 36 weeks till the tumor regresses, wherein the administration cycle comprises of the administration of NOD2 agonist to a subject in a dose range of 0.03 mg/kg to 0.2 mg/kg of body weight twice a week for 12 weeks followed by once a week for 24 weeks, and the administration of CTLA4 antagonist of about 1 to about 3 mg/kg on 7 day, once in three weeks (Q3W) and repeat the cycle till the tumor regresses wherein the subject is human.

In certain embodiments, the dosing regimen of NOD2 agonist and PD-1 axis antagonist or CTLA4 antagonist can vary over the time. For example, initially NOD2 agonist may be administered at a higher dose and gradually reduces the doses, or vice versa (i.e. starting at a lower dose and gradually increasing the doses). Similarly, PD-1 axis antagonist and CTLA4 antagonist may be administered at a higher dose and gradually reduce the doses, or vice versa (i.e. starting at a lower dose and gradually increasing the doses).

In certain embodiments, the NOD2 agonist and the PD-1 axis antagonist are administered until the clinical benefit is observed or until there is a complete response, or unmanageable toxicity or disease progression occurs.

In some embodiments, the NOD2 agonist and the PD-1 axis antagonist are administered as a first line treatment (e.g. the initial or first treatment) or as a second line treatment (after relapse of first treatment and/or first line treatment has failed).

In certain embodiments, the dosage and frequency of administration of the NOD2 agonist and the PD-1 axis antagonist can vary depending on whether the treatment is prophylactic or therapeutic. In prophylactic applications, a relatively low dosage is typically administered at relatively infrequent intervals over a long duration of time. Some subjects continue to receive treatments for the rest of their lives. In therapeutic applications, a relatively high dosage at relatively short intervals is sometimes required until progression of the disease is reduced or terminated, or until the patient shows partial or complete amelioration of symptoms of disease.

In certain embodiments, an effective dosage level of the NOD2 agonist is used. In some embodiments, the present pharmaceutical compositions can be used to obtain a therapeutically effective amount of the NOD2 agonist that is effective to achieve the desired therapeutic response, without being unduly toxic to the subject. The selected dosage level can vary depending on the pharmacokinetics factors such as the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In certain embodiments, the combination of a NOD2 agonist and an immunotherapeutic agent can be administered by the same route or different routes. In certain embodiments, the NOD2 agonist and the immunotherapeutic agent is administered intravenously, intramuscularly, subcutaneously, intratracheally, vaginally, intraperitoneally, intraorbitally, orally, transdermally, implantation, inhalation, intrathecally, intraventricularly, intranasally, or any other possible route. In some embodiments, the preferred route of administration is intravenous.

### V. Cancer/Tumors

The present disclosure provides methods of preventing or treating cancer. Mifamurtide is known to execute its antitumor effect both directly or indirectly through macrophage or monocyte activation. The activated antigen presenting cells (APCs) are cytotoxic towards tumor cells. The active component of Mifamurtide, MTP, is a specific ligand of NOD2, which is an intracellular receptor found in monocytes, dendritic cell, and macrophages. Thus, the activation of the APCs is associated with enhanced secretion of pro-inflammatory cytokines and chemokines. Therefore, this inflammatory milieu is unexpectedly found to enhance the tumor infiltration of tumoricidal immune cell and make the refractory stages of advanced cancer responsive to any immune therapy.

The present disclosure provides a combination of a NOD2 agonist and an immunotherapeutic agent, which is used preferably for the treatment of cancers or tumor including colorectal cancer, melanoma, osteosarcoma, head and neck cancer, gastric cancer, breast cancer (triple negative breast cancer), acute lymphoblastic leukaemia (ALL), non-small cell lung cancer (NSCLC), ovarian cancer, hepatocellular cancer, pancreatic cancer, renal cell cancer, and bladder cancer. The preferred cancer is colorectal cancer.

The present disclosure provides a combination of NOD2 agonist and an immunotherapeutic agent, which is used preferably for the treatment of cancers or tumor including head and neck cancer, breast cancer, non-small cell lung cancer (NSCLC), pancreatic cancer, colorectal cancer, ovarian cancer, renal cell cancer (Clear cell renal cell carcinoma, Prenatal renal cell carcinoma), bladder cancer, melanoma, acute mycloid leukemia (AML), cervical cancer, lung cancer, mesothelioma, testicular germ cell, uterine cancer, cholangiocarcinoma, liver cancer, thyoma, thyroid cancer, prostate cancer, adenoid cystic carcinoma, putative gastric progenitor cell cancer, uveal melanoma, esophagus cancer, glioma, neuroendocrine prostate cancer (NEPC), Diffuse B-cell lymphoma (DLBC), and Desmoplastic small-round-cell tumor.

A non-limiting list of cancers in which the combination therapy of the present invention is effective. Specific examples of cancer include, but are not limited to acute lymphoblastic leukemia, adrenocortical carcinoma, AIDS-related lymphoma, central nervous system lymphoma, AIDS-related malignancies, anal cancer, childhood cerebral astrocytoma, bile duct cancer, extrahepatic bile duct cancer, bladder cancer, osteosarcoma or bone cancer, fibrosarcoma, bone and connective tissue sarcomas, giant cell carcinoma, skin cancer (for e.g. squamous cell carcinoma, basal cell carcinoma, melanoma), uveal melanoma, sweat gland carcinoma, sebaceous gland carcinoma, brain tumor or glioma, glioblastoma multiform, medulloblastoma, craniopharngioma, pinealoma, hemangioblastoma, acoustic neuroma, oligodendroglioma, breast cancer (such as hormone refractory metastatic breast cancer, triple negative breast cancer), lung cancer, bronchial adenomas, carcinoid tumor, adrenocortical carcinoma, islet cell carcinoma, T-cell lymphoma, central nervous system lymphoma Diffuse B-cell lymphoma (DLBC), cervical cancer, acute lymphoblastic leukemia, chronic lymphoblastic leukemia, acute lymphocytic leukemia, chronic lymphocytic leukemia, acute myelogenous leukemia (AML), chronic myelogenous leukemia (CML), hairy cell leukemia, acute myeloid leukemia, chronic myelogenous leukemia, chronic myeloproliferative disorders, clear cell sarcoma of tendon sheaths, colon cancer, colorectal cancer, colorectal adenocarcinoma, adenoid cystic carcinoma, cutaneous T-cell lymphoma, Schwannoma, endometrial cancer, ependymoma, ovarian cancer, esophageal cancer, Ewing's family of tumors, extracranial germ cell tumor, extragonadal germ cell tumor, eye cancer, intraocular melanoma, medulloblastoma retinoblastoma, gall bladder cancer, abdominal cancer, gastric cancer, putative gastric progenitor cell cancer, gastrointestinal carcinoid tumor, extragonadal germ cell tumor, ovarian germ cell tumor, gestational trophoblastic tumor, head and neck cancer, hepatocellular cancer, Ewing's sarcoma, Hodgkin's lymphoma, Non-Hodgkin's lymphoma, an anaplastic large-cell lymphoma, hypopharyngeal cancer, Kaposi's sarcoma, Kidney cancer, laryngeal cancer, oral cancer, lip or oral cavity cancer, throat cancer, oropharyngeal cancer, liver cancer, non-small cell lung cancer (NSCLC), small cell lung cancer, Waldenstrom's macroglobulinemia, malignant mesothelioma, mesothelioma, malignant thymoma, thymoma, Merkel cell carcinoma, Metastatic squamous neck cancer with occult primary, multiple endocrine neoplasia syndrome, Desmoplastic small round cell tumor, neuroendocrine tumor, multiple myeloma/Plasma cell neoplasm, mycosis fungoides, myelodysplasia syndrome, nasal cavity and paranasal sinus cancer, nasopharyngeal cancer, neuroblastoma, neurofibroma, ovarian cancer, pancreatic cancer, parathyroid cancer, thyroid cancer, penile cancer, testicular cancer, testicular germ cell cancer, uterine cancer, urethral cancer, vaginal cancer, vulvar cancer, Pheochromocytoma, pineal and supratentorial primitive neuroectodermal tumors, pituitary tumor, prostate cancer, neuroendocrine prostate cancer (NEPC), rectal cancer, renal cell cancer, prenatal renal cell cancer, clear cell renal cell cancer, renal pelvis and ureter cancer, transitional cell cancer, Rhabdomyosarcoma, salivary gland cancer, sezary syndrome, meningioma, skin cancer, small intestine cancer, squamous neck cancer, cholangiocellular cancer, inflammatory myofibroblastic tumor (IMT), tenosynovial giant cell tumor (TGCT), or giant cell tumor of the tendon sheath (GCT-TS), cholangiocarcinoma, cystadenocarcionoma, ameloblastoma, chondrosarcoma, dermatofibrosarcoma, ganglioglioma, leiomyosarcoma, osteoblastoma, Wilm's tumor, and inoperable non-inflammatory locally advanced disease and the like. Virally associated cancers include Epstein-Barr virus (EBV), hepatitis B virus (HBV), hepatitis C virus (HCV), human papilloma viruses (HPV), human T lymphotropic virus type 1 (HTLV-1), human T lymphotropic type 2 (HTLV-2) and human herpesvirus, such as human herpesvirus 8 (HHV-8).

In a preferred embodiment, the combination of a NOD2 agonist and an immunotherapeutic agent is used for the treatment of colorectal adenocarcinoma, wherein the colorectal adenocarcinoma types include microsatellite instable high (MSI-H) colorectal adenocarcinoma, Kirsten Ras (KRAS) wild type of colorectal adenocarcinoma, Kristen Ras (KRAS) mutant type of colorectal adenocarcinoma. In some embodiments, the combination therapy is used for the treatment of the refractory cancer. Thus, the methods include administering the compositions to colorectal adenocarcinoma patients determined to have wild-type K-ras, mutant, K-ras, high (MSI-H) colorectal adenocarcinoma. The determining step may include any suitable laboratory test, for example, antibody-based analysis or nucleic-based tests (e.g., PCR).

In some embodiments, the combination of a NOD2 agonist and an immunotherapeutic agent is used for the treatment of the non-responsive or the cold type of colorectal cancer and converts the non-responsive or the cold type of colorectal cancer to the hot immunogenic colorectal cancer that responds to the immunotherapeutic agent.

In some embodiments, the combination therapy may be used prior to or following surgery to remove a tumor and may be used prior to, during or after radiation therapy, standard cancer chemotherapies, gene therapy or vaccines.

### VI. Compositions

In some embodiments, the present disclosure provides compositions comprising a NOD2 agonist and an immunotherapeutic agent (e.g. formulated together in a single composition or separately formulated).

In certain embodiments, the NOD2 agonist and the immunotherapeutic agent (e.g. PD-1 antagonist, PD-L1 antagonist. PD-L2 antagonist, and CTLA4 antagonist) can be administered in a pharmaceutical composition with pharmaceutically acceptable carriers or diluents.

In certain embodiments, the pharmaceutical compositions of the present disclosure can be suitable for administering to the subject are typically formulated for parenteral administration such as in a liquid carrier, or suitable for reconstitution into liquid solution or suspension for intravenous administration. In certain embodiments, the pharmaceutical compositions are also suitable for administration by oral, inhalational, rectal, topical, or transdermal routes.

In other embodiments, the composition comprises a pharmaceutically acceptable carrier. As used herein, the term “pharmaceutically acceptable” means approved by a government regulatory agency or listed in the U.S. Pharmacopeia or another generally recognized pharmacopeia for use in the subject, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose, sucrose, mannitol and glycerol solutions may be employed as carriers, particularly for injectable solutions.

Liquid compositions for parenteral administration can be formulated for administration by injection or continuous infusion. Routes of administration by injection or infusion can include, but are not limited to intravenous, intraperitoneal, intramuscular, intrathecal, and subcutaneous. In some embodiments, parenteral formulations can include prefilled syringes, vials, powder for infusion for reconstitution, concentrate for infusion to be diluted before delivery (ready to diluted), solutions (ready to use).

Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. For parenteral use, Mifamurtide composition may be available in a powder form for dispersion for infusion and containing the Palmitoyl-2-oleoyl-sn-glycero-3-phosphocholine (POPC), 1, 2-Dioleoyl-sn-glycero-3-phospho-L-serine monosodium salt (OOPS) as pharmaceutical excipients. Opdivo® is available as an injectable solution in a single dose vial and containing mannitol, pentenic acid, polysorbate 80, sodium chloride, sodium citrate dihydrate and water for injection as pharmaceutical excipients. Keytruda® is available as injectable solution or lyophilized powder in a single-use vial and containing L-histidine, polysorbate 80, sucrose, and water for injection as pharmaceutical excipients.

In certain embodiments, the composition of the present invention includes biodegradable subcutaneous implant, osmotically controlled device, subcutaneous implant, subcutaneous sustained release injection, lipid nano particles, liposomes, and the like. Liquid preparations can include, but are not limited to solutions, suspensions and emulsions. Such preparations are exemplified by water or water/propylene glycol solutions for parenteral injection. Liquid preparations may also include solutions for intranasal administration.

For oral use, the pharmaceutical compositions of the present invention may be administered, for example, in the form of tablet, capsule, powder, dispersible granule, cachet, aqueous solution, or suspension. In the case of tablet for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled to render the preparation isotonic. For preparing suppositories, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Aerosol preparations suitable for inhalation may include solutions and solids in powder forms, which may be in combination with a pharmaceutically acceptable carrier, such as an inert compressed gas.

Suitable formulations for transdermal applications include an effective amount of a NOD2 agonist with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Also included are solid preparations which are intended for conversion, shortly before use, to liquid preparations for either oral or parenteral administration. Such liquid forms include solutions, suspensions, and emulsions.

### VII. Kits

As used herein, “kit” refers to packaged therapeutics agents individually or in combination with immunotherapeutic agents that optionally includes other elements, such as instructions for using the elements thereof.

Also, the kits described in the present disclosure comprise a NOD2 agonist and an immunotherapeutic agent for therapeutic uses. Kits typically include a label indicating the intended use of the contents of the kit and instructions for use. The term label includes any writing, or recorded material supplied on or with the kit, or which otherwise accompanies the kit.

Accordingly, this disclosure provides a kit for treating a subject afflicted with a cancer, the kit comprising: (a) a dosage ranging from about 0.01 mg/kg to about 1.5 mg/kg of body weight of NOD2 agonist; (b) a dosage ranging from about 0.1 mg/kg to about 30 mg/kg of body weight of PD-1 axis antagonist; and (c) instructions for using the NOD2 agonist and the PD-1 axis antagonist in any of the combination therapy methods disclosed herein.

In some embodiments, the dosage of the NOD2 agonist can range from about 0.01 mg/kg to about 1.5 mg/kg of body weight, about 0.02 mg/kg to about 1 mg/kg of body weight, about 0.05 mg/kg to about 0.5 mg/kg of body weight, about 0.1 mg/kg to about 0.4 mg/kg of body weight, about 0.2 mg/kg to about 0.3 mg/kg of body weight, inclusive of all ranges and subranges therebetween. In some embodiments, the dosage of the PD-1 axis antagonist can range from about 0.1 mg/kg to about 30 mg/kg of body weight, about 0.5 mg/kg to about 25 mg/kg of body weight, about 1 mg/kg to about 20 mg/kg of body weight, about 5 mg/kg to about 15 mg/kg of body weight, about 10 mg/kg to about 13 mg/kg of body weight, about 0.3 mg/kg to about 11 mg/kg of body weight, about 17 mg/kg to about 30 mg/kg of body weight, inclusive of all ranges and subranges therebetween.

In certain embodiments, the NOD2 agonist and the PD-1 axis antagonist can be co-packaged in unit dosage form. In certain embodiments, the kit comprises NOD2 agonist disclosed herein, e.g., Mifamurtide, for treating human patients. In other embodiments, the kit comprises a PD-1 axis antagonist disclosed herein, e.g., Keytruda® or Opdivo®.

In certain embodiments, the present disclosure provides kits comprising a NOD2 agonist and an immunotherapeutic agent, for treating or delaying the progression of a cancer in a subject or enhancing immune function of the subject having cancer. The kits further comprise a package insert with instructions for administering the combination therapy either simultaneously or sequentially in a subject having cancer. In some embodiments, the kits further comprise one or more of another agent (e.g. chemotherapeutic agent).

In some embodiment, the kits comprise a combination of a NOD2 agonist (for e.g. Mifamurtide) and a PD-1 axis antagonist (for e.g. PD-1 antibody, PD-L1 antibody, PD-L2 antibody), or a CTLA4 antagonist with a package insert containing instructions for treating or delaying the progression of a cancer in a subject or enhancing immune function of a subject having cancer.

In some embodiments, the kits comprise a NOD2 agonist and a package insert comprising instructions for using the NOD2 agonist in combination of an immunotherapeutic agent to treat or delay progression of cancer in a subject or to enhance immune function of a subject having cancer. In some embodiments, the kits comprise an immunotherapeutic agent and a package insert comprising instructions for using the immunotherapeutic agent in combination of NOD2 agonist to treat or delay progression of cancer in a subject or to enhance immune function of a subject having cancer.

In some embodiments, the kits comprise a first container, a second container, and a package insert, wherein the first container comprises at least one dose of a medicament of NOD2 agonist and a second container comprises at least one dose of a medicament of an immunotherapeutic agent (e.g. PD-1 antibody, PD-L1 antibody, PD-L2 antibody, or CTLA4 antibody) and the package insert comprises instructions for treating or delaying progression of cancer in a subject or to enhance immune function of a subject having cancer, wherein the medicament comprises of any suitable dosage form for administration that known to a person skilled in the art.

The first and the second containers may be comprised of the same or different shapes (e.g., vials, syringes and bottles). The container may be formed from a variety of materials such as glass, plastic (such as polyvinyl chloride or polyolefin), or metal alloy (such as stainless steel or has telloy). The kits may further comprise other materials that may be useful in administering the medicaments, such as diluents, filters, IV bags and lines, needles and syringes.

Optionally, the kits include multiple packages of the single dose pharmaceutical compositions of the NOD2 agonist and an immunotherapeutic agent for a single administration.

In certain embodiments, the kits comprise the combination of a NOD2 agonist or an immunotherapeutic agent in a suitable dosage form (e.g. intravenous), a dosage regimen, and a package insert. Accordingly, the dosage form is described for administration in a kit is only illustrative and other dose range or combinations thereof also included in the scope of the present disclosure.

### VIII. Outcomes

The methods of the present disclosure show clinical benefits in the cancer patients by alleviating the symptoms in the patients or increasing the life expectancy of the patients. In some embodiments, the combination therapy shows improvement by reducing the quantity and/or the size of the tumor lesions. The tumor lesions can be determined by a variety of methods known in the art, which may include, but are not limited to by measuring the dimensions of tumor(s) upon removal from the subject, e.g., using caliper, or while in the body using imaging techniques, e.g., ultrasound, CT or MRI scans. In some embodiment, cytology or histology can be used to evaluate responsiveness to therapy.

In some embodiments, administering a therapeutically effective amount of a combination of a NOD2 agonist and an immunotherapeutic agent produces at least one therapeutic effect selected from the group consisting of reduction in size and/or volume of a tumor, reduction in number of metastatic lesions appearing over time, partial response, complete response or stable disease.

In certain embodiments, the clinical outcome of the patient population is analyzed by administration of the combination therapy of NOD2 agonist and an immunotherapeutic agent that showed better survival rate as compared to the patient population receiving either NOD2 agonist or an immunotherapeutic agent.

In some embodiments, the combination therapy of a NOD2 agonist and an immunotherapeutic agent shows better immune response or immune stimulation by increasing the level of proinflammatory cytokines such as IFN-γ, IL-6, IL-12p40, TNF-α or chemokines such as MCP-1, MIP-2 that is not achieved by NOD2 agonist or an immunotherapeutic agent alone.

In certain embodiments, inhibition of tumor growth can be assessed by measuring the size of tumor before or after the administration of a therapeutic combination of the present invention (e.g. administration of PD-1 antagonist and Mifamurtide). A reduction in size of the tumor, or a reduction in the rate of the tumor growth, following administration of a therapeutic combination of the present invention as compared to the size and/or growth rate of the tumor prior to administration of the therapeutic combination of the present invention indicates an inhibition of the growth of a tumor in the subject. In certain embodiments, the methods of the present invention result in tumor regression.

In some embodiments, the method of treating tumor by administration of a combination of a NOD2 agonist and an immunotherapeutic agent shows reduction of tumor growth by at least about 5%, 10%, 15%, 20%, 25%, 30%, 35%, 40%, 45%, 50%, 55%, 60%, 65%, 70%, 75%, 80%, or more compared to NOD2 agonist or immunotherapeutic agent alone. In other embodiments, the improvement in the clinical outcome is at least about 5%, 10%, 20%, 30%, 40%, 50%, 60%, 70%, 80%, or more compared to NOD2 agonist or immunotherapeutic agent alone. In some embodiments, the subject is in partial or full remission.

In certain embodiments, the combination methods result in an inhibition of tumor size more than about 10%, more than about 20%, more than about 30%, more than about 35%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 65%, more than about 70%, more than about 75%, more than about 80%, more than about 85%, more than about 90%, more than about 95%, or more than about 100%. In certain embodiments, the administration of a PD-1 axis antagonist in combination of NOD2 agonist leads to complete regression of tumor growth.

In certain embodiments, the combination of a NOD2 agonist and an immunotherapeutic agent shows enhanced therapeutic response to the reduction in the growth of the tumor. For example, enhanced response comprises an increase in responsiveness of at least about 5%, 10%, 15%, 20%, 25%, 30%, 35%, 40%, 45%, 50%, 55%, 60%, 65%, 70%, 75%, 80%, 85%, 90%, 95% or 98% or more.

In certain embodiment, the clinical outcome of the present invention depends upon the modes of administration, condition being treated, and the desired outcome. It also depends on the stage of condition, age and physical condition of the subject, prior other diseases associated with cancer, the nature of concurrent therapy, if any, and like factors known to the practitioner.

In a preferred embodiment, the method of treatment of colon adenocarcinoma by administration of Mifamurtide and PD-1 antagonist produces better clinical outcome compared to the outcomes achieved by administering Mifamurtide or PD-1 antagonist alone.

In some embodiments, the present combination therapies can also induce apoptosis in the tumor when administering to the subjects. In some embodiments, the formulations of the combination therapies can include Mifamurtide and an immunotherapeutic agent that synergistically increase the apoptosis. In some embodiments, the apoptosis caused by the combination therapies can be increased by 50%, 55%, 60%, 65%, 70%, 75%, 80%, 85%, 90%, 95%, 100%, inclusive of all ranges and subranges therebetween, compared to the apoptosis caused by either Mifamurtide or an immunotherapeutic agent alone. In some embodiments, the immunotherapeutic agent can be a PD-1 antagonist, a PD-L1 antagonist, a PD-L2 antagonist, a CTLA4 antagonist, or any suitable agent as described herein.

### IX. Specific Embodiments of the Present Invention are as Follows

The following embodiments further describe the objects of the present invention in accordance with the best mode of practice; however, disclosed invention is not restricted to the embodiments hereinafter described.

### Embodiment 1

A method for treating a subject having cancer comprising administering to the subject a combination of therapeutically effective amount of
(i) a NOD2 agonist; and
(ii) an immunotherapeutic agent.

### Embodiment 2

The method of embodiment 1, wherein the NOD2 agonist is selected from the group comprising of Murabutide, Mifamurtide, Muramyl tetrapeptide, Muramyl tripeptide, Muramyl dipeptide, Romurtide, M-TriDaP (N-acetyl-muramyl-L-Ala-γ-D-Glu-meso-diaminopimelic acid), N-Glycolyl Muramyldipeptide, M-TriLYS (MurNAc-Ala-D-isoGln-Lys), MDP(D-Glu2)-OCH3, Glucosaminyl muramyldipeptide, or any combination thereof.

### Embodiment 3

The method of embodiment 2, wherein NOD2 is administered at a dose of about 0.01 mg/kg to about 1.5 mg/kg of body weight, preferably about 0.01 mg/kg to about 0.5 mg/kg of body weight and more preferably at a dose of about 0.03 mg/kg to about 0.2 mg/kg of body weight, twice every weekly or once weekly.

### Embodiment 4

The method of embodiment 2, wherein the NOD2 agonist is Mifamurtide.

### Embodiment 5

The method of embodiment 1, wherein the immunotherapeutic agent is preferably selected from the group comprising of PD-1 axis antagonist or CTLA4 antagonist, wherein said PD-1 axis antagonist comprising of PD-1 antagonist, PD-L antagonist or PD-L2 antagonist.

### Embodiment 6

The method of embodiment 5, wherein the PD-1 antagonist is selected from group comprising of ANA011, AUNP-12, BGB-A317, KD033, Pembrolizumab. MCLA-134, mDX400, MEDI0680, muDX400, Nivolumab, PDR001, PF-06801591, REGN-2810, SHR-1210, STI-A1110, TSR-042, ANB011, or XCE853.

### Embodiment 7

The method of embodiment 6, wherein the PD-1 antagonist is Pembrolizumab or Nivolumab.

### Embodiment 8

The method of embodiment 6, wherein the PD-1 antagonist is administered at a dose of about 0.1 mg/kg to about 10 mg/kg of body weight, once every two, three or four weeks, more preferably at a dose of about 2 mg/kg to about 5 mg/kg of body weight, once every two or three weeks.

### Embodiment 9

The method of embodiment 5, wherein the PD-L1 antagonist is selected from group comprising of Avelumab, BMS-936559. CA-170, Durvalumab, MCLA-145, SP142, STI-A1011. STI-A1012, STI-A1010, STI-A1014 and Atezolimumab.

### Embodiment 10

The method of embodiment 9, wherein the PD-L1 antagonist is Durvalumab, Atezolimumab, or Avelumab.

### Embodiment 11

The method of embodiment 9, wherein the PD-L antagonist is administered at a dose of about 1 mg/kg to about 20 mg/kg of body weight once every three weeks.

### Embodiment 12

The method of embodiment 5, wherein the PD-L2 antagonist is AMP-224 or rHIgM12B7.

### Embodiment 13

The method of embodiment 12, wherein the PD-L2 antagonist is administered at a dose range of about 0.3 mg/kg to 30 mg/kg of body weight, once every two weeks.

### Embodiment 14

The method of embodiment 5, wherein the CTLA4 antagonist are selected from group comprising of KAHR-102, ABR002, KN044, Tremelimumab and Ipilimumab.

### Embodiment 15

The method of embodiment 14, wherein the CTLA4 antagonist is Tremelimumab or Ipilimumab.

### Embodiment 16

The method of embodiment 14, the CTLA4 antagonist is administered at a dose of about 1 mg/kg to about 3 mg/kg of body weight, once every three weeks.

### Embodiment 17

The method of embodiment 1, wherein said NOD2 agonist is administered intravenously.

### Embodiment 18

The method of embodiment 5, wherein the PD-1 axis antagonist or CTLA4 antagonist are administered intravenously.

### Embodiment 19

The method of embodiment 1, wherein the cancer is a refractory cancer.

### Embodiment 20

The method of embodiment 1, wherein the cancer is selected from group comprising of colorectal cancer, melanoma, osteosarcoma, head and neck cancer, gastric cancer, breast cancer (triple negative breast cancer), acute lymphoblastic leukemia (ALL), non-small cell lung cancer (NSCLC), ovarian cancer, hepatocellular cancer, pancreatic cancer, renal cell cancer, and bladder cancer.

### Embodiment 21

The method of embodiment 20, wherein the cancer is colorectal cancer.

### Embodiment 22

The method of embodiment 20, wherein the cancer is melanoma.

### Embodiment 23

The method of embodiment 20, wherein the cancer is osteosarcoma.

### Embodiment 24

The method of embodiment 20, wherein the cancer is head and neck cancer.

### Embodiment 25

The method of embodiment 20, wherein the cancer is gastric cancer.

### Embodiment 26

The method of embodiment 20, wherein the cancer is triple negative breast cancer.

### Embodiment 27

The method of embodiment 20, wherein the cancer is acute lymphoblastic leukemia.

### Embodiment 28

The method of embodiment 20, wherein the cancer is non-small cell lung cancer.

### Embodiment 29

The method of embodiment 20, wherein the cancer is ovarian cancer.

### Embodiment 30

The method of embodiment 20, wherein the cancer is hepatocellular cancer.

### Embodiment 31

The method of embodiment 20, wherein the cancer is pancreatic cancer.

### Embodiment 32

The method of embodiment 20, wherein the cancer is renal cell cancer.

### Embodiment 33

The method of embodiment 20, wherein the cancer is bladder cancer.

### Embodiment 34

The method of embodiment 1, wherein said NOD2 agonist and said immunotherapeutic agent are administered simultaneously or sequentially in either order.

### Embodiment 35

The method of embodiment 1, wherein the combination of said NOD2 agonist and said immunotherapeutic agent are administered for as long as clinical benefit is observed or until unmanageable toxicity or disease progression occurs.

### Embodiment 36

The method of embodiment 1, wherein said NOD2 agonist and said immunotherapeutic agent are administered concurrently in separate compositions.

### Embodiment 37

The method of embodiment 1, wherein the subject is a human.

### Embodiment 38

A kit for treating a subject afflicted with a cancer, the kit comprising:
(i) a dosage ranging from about 0.01 mg/kg to about 1.5 mg/kg of body weight of NOD2 agonist;
(ii) a dosage ranging from about 0.1 mg/kg to about 30 mg/kg of body weight of PD-1 axis antagonist and
(iii) a package insert for administering NOD2 agonist and PD-1 axis antagonist, either simultaneously or sequentially.

### Embodiment 39

A method of treating a subject receiving an immunotherapeutic agent for the treatment of cancer, the method comprising administering a therapeutically effective amount of a NOD2 agonist to the subject in combination with said immunotherapeutic agent, wherein the effect is to enhance or prolong the anti-cancer effects of said immunotherapeutic agent,
wherein said NOD2 agonist is Mifamurtide, and
wherein said immunotherapeutic agent is a PD-1 axis antagonist, or a CTLA4 antagonist,
wherein said PD-1 axis antagonist comprises of PD-1 antagonist, PD-L1 antagonist or PD-L2 antagonist.

### Embodiment 40

The method of embodiment 39, wherein said NOD2 agonist is administered either simultaneously or sequentially with said immunotherapeutic agent.

### Embodiment 41

The method of embodiment 39, wherein said combination comprises of:
i. said PD-1 antagonist is administered at a dose of about 0.1 mg/kg to about 10.0 mg/kg of body weight, once every two, three or four weeks, more preferably at a dose of about 2 mg/kg to about 5 mg/kg of body weight, once every two or three weeks.
ii. said Mifamurtide is administered at a dose of about 0.01 mg/kg to about 1.5 mg/kg of body weight, preferably about 0.01 mg/kg to about 0.5 mg/kg of body weight and more preferably about 0.03 mg/kg to about 0.2 mg/kg of body weight, twice every week for 12 weeks followed by once a week for 24 hours.

### Embodiment 42

The method of embodiment 39, wherein said combination comprises of:
i. said PD-L antagonist is administered at a dose of about 1 mg/kg to about 20 mg/kg of body weight once every three weeks.
ii. said Mifamurtide is administered at a dose of about 0.01 mg/kg to about 1.5 mg/kg of body weight, preferably about 0.01 mg/kg to about 0.5 mg/kg of body weight and preferably about 0.03 mg/kg to about 0.2 mg/kg of body weight, twice every week for 12 weeks followed by once a week for 24 weeks.

### Embodiment 43

The method of embodiment 39, wherein said combination comprises of:
i. said PD-L2 antagonist is at a dose of about 0.3 mg/kg to 30 mg/kg of body weight, once every three weeks.
ii. said Mifamurtide is administered at a dose of about 0.01 mg/kg to about 1.5 mg/kg of body weight, preferably about 0.01 mg/kg to about 0.5 mg/kg of body weight and more preferably about 0.03 mg/kg to about 0.2 mg/kg of body weight twice every week for 12 weeks followed by once a week for 24 weeks.

### Embodiment 44

The method of embodiment 39, wherein said combination comprises of:
i. said CTLA4 antagonist is administered at a dose of about 1 to about 3 mg/kg of body weight, once every three weeks.
ii. said Mifamurtide is administered at a dose of about 0.01 mg/kg to about 1.5 mg/kg of body weight, preferably about 0.01 mg/kg to about 0.5 mg/kg of body weight and preferably about 0.03 mg/kg to about 0.2 mg/kg of body weight, twice every week for 12 weeks followed by once a week for 24 weeks.

### Embodiment 45

A method of enhancing immune function in a subject having cancer comprises administering a therapeutically effective amount of a combination of a NOD2 agonist and an immunotherapeutic agent, wherein said NOD2 agonist is Mifamurtide and said immunotherapeutic agent comprises of PD-1 antagonist, PD-L1 antagonist, PD-L2 antagonist or CTLA4 antagonist.

### Embodiment 46

The method of embodiment 45, wherein the subject has enhanced proliferation and/or cytolytic activity relative to prior to the administration of the combination.

### Embodiment 47

A method of treating cancer comprising administering to the subject in need thereof an effective amount of Mifamurtide and an immunotherapeutic agent that modulates the activity of immuno-onco targets selected from group comprising of PD-1, PD-L1, PD-L2 and CTLA4.

### Embodiment 48

The method of embodiment 47, wherein the immunotherapeutic agent comprising of PD-1 antagonist, PD-L antagonist, PD-L2 antagonist or CTLA4 antagonist.

### Embodiment 49

The method of embodiments 39, 45, or 48 wherein the PD-1 antagonist is selected from the group comprising of ANA011, AUNP-12. BGB-A317. KD033, Pembrolizumab, MCLA-134, mDX400, MEDI0680, muDX400, Nivolumab, PDR001, PF-06801591, REGN-2810, SHR-1210, STI-A110, TSR-042, ANB011, and XCE853.

### Embodiment 50

The method of embodiments 39, 45, or 48 wherein the PD-L antagonist is selected from group comprising of Avelumab, BMS-936559, CA-170, Durvalumab. MCLA-145, SP142, STI-A1011, STI-A1012, STI-A1010. STI-A1014 and Atezolimumab.

### Embodiment 51

The method of embodiments 39, 45, or 48 wherein the PD-L2 antagonist is AMP-224 or rHIgM12B7.

### Embodiment 52

The method of embodiments 39, 45, or 48 wherein the CTLA4 antagonist is selected from group comprising of KAHR-102, ABR002, KN044, Tremelimumab and Ipilimumab.

### Embodiment 53

A pharmaceutical composition comprising a NOD2 agonist, an immunotherapeutic agent and one or more pharmaceutically acceptable carrier or adjuvant, wherein NOD2 agonist is Mifamurtide.

### Embodiment 54

The composition of embodiment 53, wherein said immunotherapeutic agent comprises of PD-1 antagonist, PD-L1 antagonist, PD-L2 antagonist or CTLA4 antagonist.

### Embodiment 55

The composition of embodiment 54, wherein the PD-1 antagonist is selected from the group comprising of ANA11, AUNP-12, BGB-A317, KD033. Pembrolizumab, MCLA-134, mDX400, MEDI0680, muDX400, Nivolumab, PDR001, PF-06801591, REGN-2810, SHR-1210, STI-A1110 TSR-042, ANB011, and XCE853.

### Embodiment 56

The composition of embodiment 54, wherein the PD-L1 antagonist is selected from the group comprising of Avelumab, BMS-936559, CA-170. Durvalumab, MCLA-145, SP142, STI-A1011, STI-A1012, STI-A1010 STI-A1014 and Atezolimumab.

### Embodiment 57

The composition of embodiment 54, wherein the PD-L2 antagonist is AMP-224 or rHIgM12B7.

### Embodiment 58

The composition of embodiment 53, wherein the CTLA4 antagonist are selected from the group comprising of KAHR-102, ABR002, KN044, Tremelimumab and Ipilimumab.

### Embodiment 59

A combination therapy for the treatment of cancer comprising of Mifamurtide and an immunotherapeutic agent comprising PD-1 axis antagonist or CTLA4 antagonist wherein said PD-1 axis antagonist comprises of PD-1 antagonist, PD-L1 antagonist or PD-L2 antagonist.

### Embodiment 60

The combination therapy of embodiment 59, wherein the PD-1 antagonist is selected from the group comprising of ANA011, AUNP-12, BGB-A317, KD033, Pembrolizumab. MCLA-134. mDX400, MEDI0680, muDX400, Nivolumab, PDR001, PF-06801591, REGN-2810, SHR-1210, STI-A1110, TSR-042, ANB011, and XCE853.

### Embodiment 61

The combination therapy of embodiment 59, wherein the PD-L antagonist is selected from the group comprising of Avelumab, BMS-936559, CA-170, Durvalumab, MCLA-145, SP142, STI-A1011, STI-A1012, STI-A1010, STI-A1014, and Atezolimumab.

### Embodiment 62

The combination therapy of embodiment 59, wherein the PD-L2 antagonist is AMP-224 or rHIgM12B7.

### Embodiment 63

The combination therapy of embodiment 59, wherein the CTLA4 antagonist is selected from the group comprising of KAHR-102. ABR002, KN044, Tremelimumab, and Ipilimumab.

### Embodiment 64

A combination therapy for the treatment of colorectal cancer comprising of Mifamurtide and an immunotherapeutic agent comprising of Nivolumab, Pembrolizumab, or Ipilimumab.

### Embodiment 65

A combination therapy for the treatment of melanoma comprising of Mifamurtide and an immunotherapeutic agent comprising of Nivolumab, Pembrolizumab, or Ipilimumab.

### Embodiment 66

A combination therapy for the treatment of osteosarcoma comprising of Mifamurtide and an immunotherapeutic agent comprising of Nivolumab, Pembrolizumab, or Ipilimumab.

### Embodiment 67

A combination therapy for the treatment of head and neck cancer comprising of Mifamurtide and an immunotherapeutic agent comprising of Nivolumab, Pembrolizumab, or Ipilimumab.

### Embodiment 68

A combination therapy for the treatment of gastric cancer comprising of Mifamurtide and an immunotherapeutic agent comprising of Nivolumab, Pembrolizumab, or Ipilimumab.

### Embodiment 69

A combination therapy for the treatment of triple negative breast cancer comprising of Mifamurtide and an immunotherapeutic agent comprising of Nivolumab, Pembrolizumab, or Ipilimumab.

### Embodiment 70

A combination therapy for the treatment of acute lymphoblastic leukemia comprising of Mifamurtide and an immunotherapeutic agent comprising of Nivolumab, Pembrolizumab, or Ipilimumab.

### Embodiment 71

A combination therapy for the treatment of non-small cell cancer comprising of Mifamurtide and an immunotherapeutic agent comprising of Nivolumab, Pembrolizumab, or Ipilimumab.

### Embodiment 72

A combination therapy for the treatment of ovarian cancer comprising of Mifamurtide and an immunotherapeutic agent comprising of Nivolumab, Pembrolizumab, or Ipilimumab.

### Embodiment 73

A combination therapy for the treatment of hepatocellular cancer comprising of Mifamurtide and an immunotherapeutic agent comprising of Nivolumab, Pembrolizumab, or Ipilimumab.

### Embodiment 74

A combination therapy for the treatment of pancreatic cancer comprising of Mifamurtide and an immunotherapeutic agent comprising of Nivolumab, Pembrolizumab, or Ipilimumab.

### Embodiment 75

A combination therapy for the treatment of renal cell cancer comprising of Mifamurtide and an immunotherapeutic agent comprising of Nivolumab, Pembrolizumab, or Ipilimumab.

### Embodiment 76

A combination therapy for the treatment of bladder cancer comprising of Mifamurtide and an immunotherapeutic agent comprising of Nivolumab, Pembrolizumab, or Ipilimumab.

### Embodiment 77

A method of enhancing proinflammatory cytokines production in a human having tumor, comprising administering therapeutic effective amounts of (i) Mifamurtide and (ii) an immunotherapeutic agent to a human having a tumor, wherein the combination of the Mifamurtide and the immunotherapeutic agent provide a synergistic increase in proinflammatory cytokines production, wherein said immunotherapeutic agent is PD-1 antagonist, PD-L1 antagonist, PD-L2 antagonist or CTLA4 antagonist.

### Embodiment 78

A method of inducing apoptosis in a tumor in a human having tumor, comprising administering therapeutic effective amounts of (i) Mifamurtide and (ii) an immunotherapeutic agent to a human having a tumor, wherein the combination of the Mifamurtide and the immunotherapeutic agent provide a synergistic increase in apoptosis, wherein said immunotherapeutic agent is PD-1 antagonist, PD-L antagonist, PD-L2 antagonist or CTLA4 antagonist.

### X. Examples

### Example 1

### Materials and Methods:

### Animals

Six to seven-week-old female C57BL/6 mice (20±4 g weight) were used in the studies. Mice received food and water ad libitum. The study protocol, the procedures involving the care and use of animals were reviewed and approved by the Institutional Animal Care and Use Committee (IACUC) to ensure compliance with the regulations of the Association for Assessment and Accreditation of Laboratory Animal Care (AAALAC).

### Reagents

DMEM medium (Cat. No.: 11960-044). Glutamax™ (Cat. No.: 35050061), Trypsin-EDTA (0.25%) (Cat. No.: 25200-056), Penicillin-Streptomycin (Cat. No.: 15070-063), Hanks' Balanced Salt solution (Cat. No.: 14175-095) were procured form Gibco, while Fetal Bovine Serum (FBS) Cat. No.: 004-001-1A was purchased from Biological Industries. PD-1 antagonist (Cat. No.: BE0146) was supplied by BioXcell. Stock solutions of PD-1 antagonist at 1 mg/ml were kept at 4° C. prior to use. Dosing solutions of PD-1 antagonist were prepared freshly before every administration in sterile phosphate buffered saline (pH 7.0) and maintained at 4° C. The test article Mifamurtide was procured from Sigma (SML0195) and prepared freshly as a working solution at concentration of 300 μg/ml and 100 μg/ml in sterile phosphate buffered saline (pH 7.0), to be administered at a total dose of 30 μg (corresponds to 1.5 mg/kg in mice) and 10 μg (corresponds to 0.5 mg/kg in mice) respectively, into the animal as indicated in Table 1. Luminex assay kit: MCYTOMAG-70K-32 was commercially available from Millipore, was used to analyze the cytokines and chemokines in the serum samples.

### Tumor Model

MC38 mouse colon cancer cell line as provided by GenScript were maintained as monolayer culture in DMEM supplemented with 10% fetal bovine serum (FBS), 1% Glutamax™ and 1% Penicillin-Streptomycin at 37° C. in an atmosphere with 5% CO₂. The cells were routinely sub-cultured every 2 days to maintain growth at exponential phase. The tumor cells growing in exponential growth phase were harvested by trypsinization, followed by centrifugation at 335×g relative centrifugal force (RCF) in a centrifuge. The supernatant was subsequently removed by aspiration. Cell pellet was re-suspended in approximately 10× volume of cell culture medium and counted. The cell suspension was centrifuged again and processed as above and finally re-suspended in HBSS at a density of 1×10⁷ cells per ml. Cell viability was determined to be ≥95% by trypan blue staining. Cell suspensions were implanted in the subcutaneous space of the flank of female C57BL/6 mice (2.0×10⁶ MC-38 cells in 0.2 ml Hanks Balanced Salt Solution). Fifty animals were inoculated subcutaneously in the right lower flank (near the dorsal thigh region) with a single volume of 0.1 ml cell suspension containing about 1×10⁶ cells.

Tumor size and body weights were measured twice weekly.

Tumor size was measured twice per week in 2 dimensions using a caliper (recorded up to one decimal point). Tumor volume, expressed in mm³, was calculated using the following formula, in which “a” and “b” were the long and the short diameters of a tumor, respectively.

V (mm³)=(a×b²)/2

Fifty (50) animals were weighed and randomized.

### Statistical Analysis

Data related to tumor volume and percent tumor growth inhibition (% TGI) were presented as mean and the standard error of the mean (SEM). Statistical analyses were conducted using Student's t-test. P<0.05 was considered statistically significant. ★ indicate P<0.05.

Six day's post tumor implant, mice were sorted into five groups of 10 mice with a mean tumor volume of ˜120 mm³. This was followed by the analysis of the antitumor effect of Mifamurtide at doses of 10 μg (b.i.w.) and 30 μg (b.i.w.) in the presence of PD-1 antagonist at a fixed dose of 5 mg/kg (b.i.w.) in this MC-38 (murine colon) tumor bearing mice. Further, the antitumor effect of Mifamurtide alone at a dose of 30 μg (b.i.w.) and as well as PD-1 antagonist at 5 mg/kg (b.i.w) respectively was also tested. The details of the dosing schedule have been in accordance to Table 1.

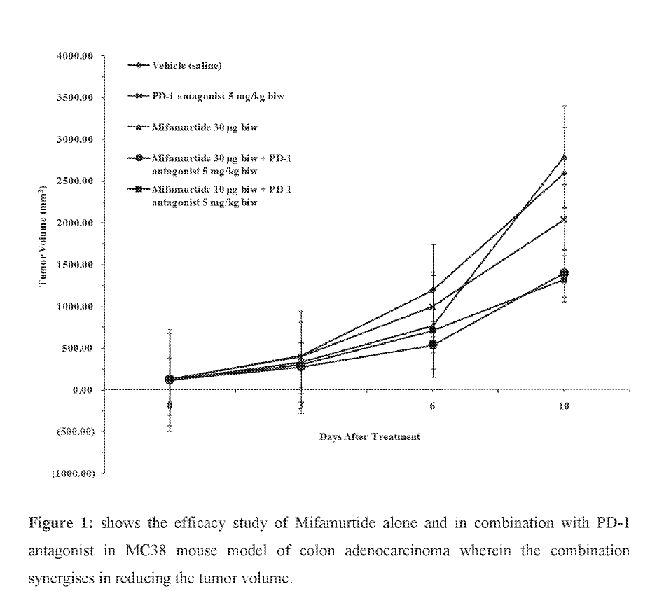

The immuno-modulatory effect of Mifamurtide at dose of 30 μg (b.i.w.) in the presence and absence of PD-1 antagonist at dose 5 mg/kg (b.i.w.) was also analysed. For this the blood was collected for analyzing serum after first dosing as per the study indicated in Table 3. Luminex analysis was done to check the levels of cytokines (IFN-γ, IL-6, IL-12p40, and TNF-α) and chemokines (MCP-1 and MIP-2). Further, since the animals within the control groups did not survive beyond day 12, unlike the animals in the combination group, the data provided herein is based on the analysis on day 10 as per FIG. 1 and Table 2 and in Table 3 at the indicated time points. Although all Mifamurtide doses and combinations were tolerated with no early death and sustained effect on body weight (data not shown).

TABLE 1
Treatment groups and dosing schedule.

| | | | Dose | | Dosing volume | | Treatment | | Route of | |
|---|---|---|---|---|---|---|---|---|---|---|
| | | | | PD-1 | Mifamurtide/ | PD-1 | frequency | | administration | |
| | No. of | | Mifamurtide | antagonist | vehicle | antagonist | Mifamurtide/ | PD-1 | Mifamurtide/ | PD-1 |
| Group | Mice | Test article | (μg) | (mg/Kg) | (ml) | (ml/Kg) | vehicle | antagonist | vehicle | antagonist |
| 1 | 10 | Vehicle (saline) | — | — | 0.2 | — | biw | — | p.o. | — |
| 2 | 10 | PD-1 antagonist | — | 5 | — | 5 | — | biw | — | i.p. |
| 3 | 10 | Mifamurtide | 30 | — | 0.1 | — | biw | — | i.p. | — |
| 4 | 10 | Mifamurtide + PD- | 30 | 5 | 0.1 | 5 | biw | biw | i.p. | i.p. |
| | | 1 antagonist | | | | | | | | |
| 5 | 10 | Mifamurtide + PD- | 10 | 5 | 0.1 | 5 | biw | biw | i.p. | i.p. |
| | | 1 antagonist | | | | | | | | |

TABLE 2
Tumor volumes and percent tumor growth inhibition (% TGI) at 10 days after administering Mifamurtide
and PD-1 antagonist as single agent or in combination in MC38 mouse model of colon adenocarcinoma.

| | | | | | | | Mifamurtide | | Mifamurtide | |
|---|---|---|---|---|---|---|---|---|---|---|
| | | | | | | | (10 μg) biw + | | (30 μg) biw + | |
| | | | | | PD-1 | | PD-1 | | PD-1 | |
| | Vehicle | | Mifamurtide | | antagonist | | antagonist | | antagonist | |
| Test article | (Saline) | | (30 μg), biw | | (5 mg/kg), biw | | (5 mg/kg), biw | | (5 mg/kg), biw | |
| Tumor | 2588.21 | (±427.83) | 2791.64 | (±649.05) | 2037.68 | (±590.74) | 1317.81 | (±297.81) | 1393.07 | (±263.83) |
| volume (mm³) | | | | | | | | | | |
| (±SEM) | | | | | | | | | | |
| TGI % | 0.00 | (±17.09) | −8.36 | (±25.95) | 22.31 | (±23.69) | 51.37* | (±11.76) | 48.42* | (±10.57) |
| (±SEM) (with | | | | | | | | | | |
| ref. to vehicle) | | | | | | | | | | |
| TGI % & | 7.79 | (±15.77) | 0.00 | (±23.95) | 28.31 | (±21.86) | 55.12 | (±10.85) | 52.40 | (±9.76) |
| SEM (with | | | | | | | | | | |
| ref. to | | | | | | | | | | |
| Mifamurtide) | | | | | | | | | | |
| TGI % | −28.62 | (±22.00) | −39.48 | (±33.41) | 0.00 | (±30.49) | 37.40 | (±15.13) | 33.61 | (±13.61) |
| (±SEM) (with | | | | | | | | | | |
| ref. to PD-1 | | | | | | | | | | |
| Antagonist) | | | | | | | | | | |

*Indicates p < 0.05
TGI = Tumor Growth Inhibition (%);
Note:
Mean (±SEM) tumor volume (mm³) measured on day 10.

TABLE 3
Luminex data for Cytokine and Chemokine profiles at
indicated time points after administering Mifamurtide
and PD-1 antagonist as single agent or in combination
in MC38 mouse model of colon adenocarcinoma.

| | IFN gamma (picogram/ml) | | |
|---|---|---|---|
| | | Post | Fold |
| Treatment | Pre | (8 hours) | change |
| Vehicle (saline) | 0 | 0.63 | 0.63 |
| PD1-antagonist, 5 mg/kg | 0 | 0 | 0.0 |
| Mifamurtide 30 μg | 0 | 0 | 0.0 |
| Mifamurtide 30 μg; PD-1 antagonist 5 mg/kg | 0 | 1.34 | 1.34 |
| | IL-6 (picogram/ml) | | |
| | | Post | Fold |
| Treatment | Pre | (8 hours) | change |
| Vehicle (saline) | 8.47 | 42.46 | 5.06 |
| PD1-antagonist, 5 mg/kg | 4.30 | 8.81 | 2.05 |
| Mifamurtide 30 μg | 1.33 | 14.43 | 10.85 |
| Mifamurtide 30 μg; PD-1 antagonist 5 mg/kg | 2.38 | 91.70 | 38.53 |
| | MCP-1 (picogram/ml) | | |
| | | Post | Fold |
| Treatment | Pre | (8 hours) | change |
| Vehicle (saline) | 13.24 | 26.44 | 2.0 |
| PD1-antagonist, 5 mg/kg | 5.12 | 5.57 | 1.1 |
| Mifamurtide 30 μg | 15.9 | 23.54 | 1.5 |
| Mifamurtide 30 μg; PD-1 antagonist 5 mg/kg | 10.14 | 112.72 | 11.12 |
| | TNF-alpha (picogram/ml) | | |
| | | Post | Fold |
| Treatment | Pre | (8 hours) | change |
| Vehicle (saline) | 1.51 | 1.95 | 1.3 |
| PD1-antagonist, 5 mg/kg | 1.82 | 1.62 | 0.9 |
| Mifamurtide 30 μg | 2.13 | 3.33 | 1.6 |
| Mifamurtide 30 μg; PD-1 antagonist 5 mg/kg | 2.37 | 3.73 | 1.6 |
| | IL-12p40 (picogram/ml) | | |
| | | Post | Fold |
| Treatment | Pre | (Day 11) | change |
| Vehicle (saline) | 1.60 | 2.37 | 1.5 |
| PD1-antagonist, 5 mg/kg | 2.27 | 1.55 | 0.7 |
| Mifamurtide 30 μg | 1.55 | 1.55 | 1.0 |
| Mifamurtide 30 μg; PD-1 antagonist 5 mg/kg | 2.58 | 34.65 | 13.43 |
| | MIP-2 (picogram/ml) | | |
| | | Post | Fold |
| Treatment | Pre | (Day 11) | change |
| Vehicle (saline) | 18.20 | 11.95 | 0.7 |
| PD1-antagonist, 5 mg/kg | 17.72 | 13.20 | 0.75 |
| Mifamurtide 30 μg | 20.84 | 13.67 | 0.7 |
| Mifamurtide 30 μg; PD-1 antagonist 5 mg/kg | 16.03 | 67.27 | 4.2 |

### Result and Observations:

Reduction in Tumor Growth:

Considering tumor volume at day 10 after treatment, Mifamurtide alone at dose of 30 μg (b.i.w.) did not show (˜−8%) any effect as compared with the vehicle control group. PD-1 antagonist at dose of 5 mg/kg (b.i.w.) showed efficacy in reducing the tumor burden, but was not substantial (only ˜22%) as compared to vehicle control as shown in FIG. 1 and Table 2. Combination of Mifamurtide at doses of 10 μg (b.i.w.) or 30 μg (b.i.w.), in the presence of PD-1 antagonist at a fixed dose of 5 mg/kg (b.i.w.) showed ˜50% Tumor Growth Inhibition (TGI) with (p<0.05) as compared to the vehicle control group on day 10 as shown in FIG. 1 and Table 2.

Cytokine and Chemokine Mediated Immuno-Stimulation:

The immunomodulation brought about by Mifamurtide showed synergistic effect when combined with PD-1 antagonist as observed in the up-regulation in terms of fold change of each pro-inflammatory cytokines including IFN-γ (Table 3), IL-6 (Table 3) and TNF-α (Table 3) at 8 hours while for IL-12p40 (Table 3) the effect was observed on day 11. Similarly, the chemokines MCP-1 (Table 3) showed enhancement at 8 hours while MIP-2 (Table 3) on day 11 indicated synergistic enhancement due to the combination. It should be noted that these are the cytokines and chemokines that curtail the immunosuppressive microenvironment and enhance the infiltration of tumoricidal monocytes and macrophages. Further, in this study it is observed that the PD-1 antagonist alone was unable to cause this immune-stimulation, as that achieved by the present combination.

Conclusion and Inference:

As shown, the present combination therapies potently caused tumor regression (FIG. 1 and Table 2) at doses of Mifamurtide 10 or 30 μg (b.i.w.) when combined with the fixed dose of PD-1 antagonist (5 mg/kg) as compared to Mifamurtide and PD-1 antagonist alone. Also, the present combination produces the immune stimulation at the same doses as indicated by the cytokine (IFN-γ, IL-6, IL-12p40, TNF-α) and chemokine (MCP-1 and MIP-2) levels, explaining that the combination of Mifamurtide with the PD-1 antagonist can synergistically enhance the immunogenicity of the poorly immunogenic tumor like colon adenocarcinoma (MC38) responsive towards PD-1 antagonism. Thereby, the current experiments indicate the phenomenon of PD-L1 upregulation in vivo, as demonstrated by the tumor regression and induction of pro-inflammatory cytokines in an immune suppressive MC38 Colon carcinoma model.

Therefore, the combination of a NOD2 agonist and a PD-1 axis antagonist can provide an effective treatment for cancer/tumor or can be used to delay the progression of the tumor/cancer. Further, it should also be noted that the presence of a NOD2 agonist during the treatment of a tumor which has low or no responsiveness to a PD 1 antagonist could transform tumors of similar kind to a responsive state through immune-simulation. For optimizing the treatment, further investigation may be required.

### INCORPORATION BY REFERENCE

All references, articles, publications, patents, patent publications, and patent applications cited herein are incorporated by reference in their entireties for all purposes.

1. A method for treating a subject having cancer comprising administering to the subject a combination of therapeutically effective amount of
(i) a NOD2 agonist; and
(ii) an immunotherapeutic agent.

2. The method of claim 1, wherein the NOD2 agonist is selected from the group consisting of Murabutide, Mifamurtide, Muramyl tetrapeptide, Muramyl tripeptide, Muramyl dipeptide, Romurtide, M-TriDaP (N-acetyl-muramyl-L-Ala-γ-D-Glu-meso-diaminopimelic acid), N-Glycolyl Muramyldipeptide, M-TriLYS (MurNAc-Ala-D-isoGln-Lys), MDP(D-Glu²)-OCH₃, Glucosaminyl muramyldipeptide, and any combinations thereof.

3. The method of claim 1, wherein NOD2 agonist is administered at a dose of about 0.01 mg/kg to about 1.5 mg/kg of body weight, preferably about 0.01 mg/kg to about 0.5 mg/kg of body weight and more preferably at a dose of about 0.03 mg/kg to about 0.2 mg/kg of body weight, twice every weekly or once weekly.

4. The method of claim 2, wherein the NOD2 agonist is Mifamurtide.

5. The method of claim 1, wherein the immunotherapeutic agent comprises a PD-1 axis antagonist or a CTLA4 antagonist, and wherein the PD-1 axis antagonist comprises a PD-1 antagonist, a PD-L1 antagonist, or a PD-L2 antagonist.

6. The method of claim 5, wherein the PD-1 antagonist is selected from the group consisting of ANA011, AUNP-12, BGB-A317, KD033, Pembrolizumab, MCLA-134, mDX400, MEDI0680, muDX400, Nivolumab, PDR001, PF-06801591, REGN-2810, SHR-1210, STI-A1110, TSR-042, ANB011, and XCE853.

7. The method of claim 6, wherein the PD-1 antagonist is Pembrolizumab or Nivolumab.

8. The method of claim 6, wherein the PD-1 antagonist is administered at a dose of about 0.1 mg/kg to about 10 mg/kg of body weight once every two, three or four weeks, more preferably at a dose of about 2 mg/kg to about 5 mg/kg of body weight once every two or three weeks.

9. The method of claim 5, wherein the PD-L1 antagonist is selected from the group consisting of Avelumab, BMS-936559, CA-170, Durvalumab, MCLA-145, SP142, STI-A1011, STI-A1012, STI-A1010, STI-A1014, and Atezolizumab.

10. The method of claim 9, wherein the PD-L1 antagonist is Durvalumab, Atezolizumab, or Avelumab.

11. The method of claim 9, wherein the PD-L1 antagonist is administered at a dose of about 1 mg/kg to about 20 mg/kg of body weight once every three weeks.

12. The method of claim 5, wherein the PD-L2 antagonist is AMP-224 or rHIgM12B7.

13. The method of claim 12, wherein the PD-L2 antagonist is administered at a dose of about 0.3 mg/kg to about 30 mg/kg of body weight, once every two weeks.

14. The method of claim 5, wherein the CTLA4 antagonist is selected from the group consisting of KAHR-102, ABR002, KN044, Tremelimumab, and Ipilimumab.

15. The method of claim 14, wherein the CTLA4 antagonist is Tremelimumab or Ipilimumab.

16. The method of claim 14, wherein the CTLA4 antagonist is administered at a dose of about 1 mg/kg to about 3 mg/kg of body weight, once every three weeks.

17. The method of claim 1, wherein the NOD2 agonist is administered intravenously.

18. The method of claim 5, wherein the PD-1 axis antagonist or CTLA4 antagonist is administered intravenously.

19. (canceled)

20. The method of claim 1, wherein the cancer is selected from the group consisting of colorectal cancer, melanoma, osteosarcoma, head and neck cancer, gastric cancer, breast cancer (triple negative breast cancer), acute lymphoblastic leukaemia (ALL), non-small cell lung cancer (NSCLC), ovarian cancer, hepatocellular cancer, pancreatic cancer, renal cell cancer, and bladder cancer.

21. The method of claim 20, wherein the cancer is colorectal cancer.

22-24. (canceled)

25. The method of claim 1, wherein the subject is human.

26. A kit for treating a subject afflicted with a cancer, the kit comprising:
(i) a dosage ranging from about 0.01 mg/kg to about 1.5 mg/kg of body weight of a NOD2 agonist;
(ii) a dosage ranging from about 0.1 mg/kg to about 30 mg/kg of body weight of a PD-1 axis antagonist; and
(iii) a package insert for either simultaneously or sequentially administering the NOD2 agonist and the PD-1 axis antagonist.

27-28. (canceled)
